(19) United States
(12) Patent Application Publication
(10) Pub. No.: US 2014/0315991 A1
(43) Pub. Date: Oct. 23, 2014

(54) COMPOSITION FOR TREATMENT OF EPITHELIAL TISSUE

(71) Applicant: NOVODERMIX INTERNATIONAL LIMITED, ROAD TOWN (VG)

(72) Inventors: Nathan Andrew SHAPIRA, Atlanta, GA (US); Amir BARZILAY, Kochav Yair (IL)

(21) Appl. No.: 14/318,821

(22) Filed: Jun. 30, 2014

Related U.S. Application Data
(63) Continuation-in-part of application No. 13/091,165, filed on Apr. 21, 2011, now abandoned.
Application No. 13/091,165 is a continuation of application No. PCT/IB2009/054646, filed on Oct. 21, 2009.
(60) Provisional application No. 61/136,994, filed on Oct. 21, 2008.

(51) Int. Cl. A61K 31/357; A61K 9/107

(52) U.S. Cl. CPC A61K 31/357; A61K 9/107
USPC 514/454

(57) ABSTRACT

A pharmaceutical composition is provided. The composition is a topical oil-in-water formulation which includes a GABA agonist such as topiramate as the active ingredient.

### RELATED APPLICATIONS

This application is a continuation-in-part of U.S. patent application Ser. No. 13/091,165 filed Apr. 21, 2011, which is a continuation of PCT Patent Application No. PCT/IB2009/054646 having International Filing Date of Oct. 21, 2009, which claims the benefit of priority under 35 USC §119(e) of U.S. Provisional Patent Application No. 61/136,994 filed Oct. 21, 2008. The contents of the above applications are all incorporated by reference as if fully set forth herein in their entirety.

### FIELD AND BACKGROUND OF THE INVENTION

The present invention relates to a composition for treating disorders associated with epithelial tissue and specifically to Topiramate compositions which are formulated for topical treatment of wounds and scars.

The skin and other epithelial tissues posses an ability to shed old cells and create new ones and as to repair, to some extent, cuts, bruises, wounds, surgical incisions and trauma incisions. However, this repair process also results in scaring. While a visible scar is an inevitable end to the healing process, the results vary with the individual. Some visible scars partially fade and improve in appearance within weeks or months from skin tissue interruption, while others remain as evidence of injury for decades.

The skin can also atrophy as a result of thinning of the epidermis and/or dermis layers. Transient and permanent skin atrophy can result from aging, congenital skin diseases, acute skin diseases, chronic skin diseases, inflammatory skin diseases, skin barrier diseases, dermatological diseases scarring, trauma scarring, surgical scarring, steroids treatment and Striae.

Simple tissues such as fat, connective tissue, and epithelium regenerate, but the skin, being a complex organ derived from 2 germ layers, heals by the formation of a predominantly fibrous tissue, i.e., a scar. If the injury sections or destroys the papillary layer of the stratum corneum, a scar will form and sometimes with disfiguring consequences (Dunkins et al. Plast Reconstr Surg. 2007 May; 119(6):1722-32).

Examples of disfiguring scars include depressed scars, irregular flat scars, widened scars, hypertrophied scars and keloid scars. Both keloid and hypertrophic scars are wounds that heal overzealously above the uninterrupted skin surface. The difference between a keloid and a hypertrophied scar is that a keloid scar continues to enlarge beyond the original size and shape of the wound, while a hypertrophied scar enlarges within the confines of the original wound. Although both can be red and raised, keloids continue to grow and hypertrophied scars tend to regress over time. Both can recur after surgical excision; however, the recurrence of keloid scars is more common Widened scars are wounds that separate during the healing process, usually in response to tension perpendicular to the wound edges.

There are some techniques that can be employed to improve the appearance of a scar, though in time, all scars improve on their own to some degree. Once a scar has matured (typically within 9-15 months), it most likely won't undergo any more changes. Surgery, chemical peeling and thermal skin ablation technologies such lasers, RF and plasma can sometimes help to partially diminish a scar. Injections of Triamcinolone, a medication which inhibits production of the collagen that makes up scar tissue, reduces inflammation and can also help a scar to regress. Further, injection of skin augmentation fillers (e.g. Collagen, Hyaluronic Acid, fat, etc.) into an atrophic scar site can also temporarily improve the appearance of such scar.

There are also topical products marketed for improving the appearance of scars when applied immediately or shortly after injury/incision. Two of these are Dermatix™, an inert silicone gel and Mederma™ which incorporates onion extract. New approaches for treating scars are also currently investigated, these include the experimental drug Juvista™ which includes human recombinant TGFβ3; clinical trials have shown that this drug, when injected into the surgical incision site immediately following surgical wound suturing/gluing, improves scar appearance in the skin.

Due to ease of use and lower risks of side effects compared to surgical excision, steroid injections, pressure therapy, thermal ablation and cryotherapy treatments, topical therapy of wounds, scarring and other skin atrophies is preferred. To date, topical therapy involving inert silicone gel or inert silicone sheets is considered the most effective in reducing scarring immediately or shortly after injury/incision although the effect of such treatment is considered less than optimal.

While reducing the present invention into practice, the present inventors have uncovered that topical formulations of a GABA agonist, and specifically oil-in-water based formulations of Topiramate are effective in reducing the healing time of excisional and incisional wounds while concurrently reducing or eliminating subsequent fresh scarring. In addition, topical Topiramate formulations were also found effective in improving the state of fresh dermatological scarring, mature dermatological scarring, fresh surgical atrophic scarring, mature surgical atrophic scarring, congenital atrophic dermatological diseases, acquired atrophic dermatological diseases, acute atrophic dermatological diseases, skin barrier diseases, autoimmune skin diseases, steroids-induced skin atrophy and Striae, and, skin-aging related disorders.

### SUMMARY OF THE INVENTION

According to one aspect of the present invention there is provided a pharmaceutical composition comprising a GABA agonist and an oil-in-water carrier.

According to further features in preferred embodiments of the invention described below, the GABA agonist is Topiramate.

According to still further features in the described preferred embodiments the pharmaceutical composition includes 0.1-7.5% (w/w) of the Topiramate.

According to still further features in the described preferred embodiments the pharmaceutical composition includes 0.5-5.0% (w/w) of the Topiramate.

According to still further features in the described preferred embodiments the oil-in-water carrier is formulated as a cream, a gel cream, an emulsion and a foam.

According to still further features in the described preferred embodiments the cream includes water, white soft paraffin, cetosteary alcohol, liquid paraffin and sodium lauryl sulfate.

According to still further features in the described preferred embodiments the cream includes water, dimethicone, stearic acid, isopropyl myristate, mineral oil, glycerin, glyceryl stearate, cetyl alcohol, pentenol and TEA.

According to still further features in the described preferred embodiments the oil-in-water carrier includes a water soluble polymer such as sclerotium gum, xanthan gum, sodium alginate, carbomer, cellulose ether or acrylate polymer.

According to still further features in the described preferred embodiments the foam includes water, mineral oil, isopropyl myristate, MCT oil, glyceryl monostreate, strearyl alcohol, xantan gum, methocel K1000M, TWEEN 80, MYRJ 49p, Glycofurol, cocoamidopropylbethaine, phenonip, butane.

According to still further features in the described preferred embodiments the GABA agonist is Topiramate.

According to another aspect of the present invention there is provided a pharmaceutical composition comprising a GABA agonist formulated for corneal application.

According to yet another aspect of the present invention there is provided a method of treating a disorder associated with epithelial tissue comprising topically applying a pharmaceutical composition comprising a GABA agonist and an oil-in-water carrier to the epithelial tissue thereby treating the disorder.

According to still further features in the described preferred embodiments the oil-in-water carrier is formulated as a cream, a gel cream an emulsion or a foam.

According to still further features in the described preferred embodiments the epithelial tissue is skin.

According to still further features in the described preferred embodiments the disorder is a wound.

According to still further features in the described preferred embodiments the disorder is a skin barrier disorder.

According to still further features in the described preferred embodiments the disorder is a combination of an autoimmune disorder and a skin barrier disorder.

According to still further features in the described preferred embodiments the disorder is a combination of an inflammatory disorder and a skin barrier disorder.

According to still further features in the described preferred embodiments the disorder is a scar.

According to still further features in the described preferred embodiments the disorder is caused by skin atrophy.

According to still further features in the described preferred embodiments the disorder is Striae.

According to still further features in the described preferred embodiments the GABA agonist is topiramate.

According to still further features in the described preferred embodiments the pharmaceutical composition includes 0.1-7.5% (w/w) of the topiramate.

According to still further features in the described preferred embodiments the scar is a depressed scar, atrophic scar, flat scar, hypertrophic scar or keloid scar.

According to still further features in the described preferred embodiments the disorder is a wound and the pharmaceutical composition is first applied 0-8 days following wounding.

According to still further features in the described preferred embodiments, the disorder is selected from the group consisting of wrinkles, warts, skin sags, cellulite and stretch marks.

According to still another aspect of the present invention there is provided an article-of-manufacturing comprising a pharmaceutical composition including topiramate and an oil-in-water carrier and packaging material identifying the pharmaceutical composition for use in treatment of disorders associated with epithelial tissue.

According to still another aspect of the present invention there is provided a method of reducing the appearance of a scar or minimizing scar formation comprising topically applying a pharmaceutical composition comprising Topiramate to tissue having a wound or scar.

According to still another aspect of the present invention there is provided a pharmaceutical composition comprising a GABA agonist formulated as a cream, a gel cream, an emulsion or a foam.

According to still further features in the described preferred embodiments the GABA agonist is Topiramate.

According to still further features in the described preferred embodiments the pharmaceutical composition includes 0.10-7.5% (w/w) of the Topiramate.

The present invention successfully addresses the shortcomings of the presently known configurations by providing a topical formulation suitable for treating skin disorders such as wounds, scars, skin atrophies and the like.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 illustrates excisional wounding of rabbit ears.

FIG. 2 illustrates markings of scars of healed wounds (black) and reference unwounded, untreated skin (green) prior to harvesting.

FIG. 3 is a histology slide of healed scar tissue obtained from wound L02 of Rabbit 10 at Day 28 of treatment.

FIGS. 4a-b illustrate measurement and calculation of cross-sectional scar area and adjacent skin area for SEI (Scar Elevation Index) Calculation. FIG. 4a is a slide of scar tissue obtained from the left ventral ear—wound L02 of Rabbit 01 on Day 28, while FIG. 4b is a slide of tissue obtained from the left ventral ear—reference skin site L-B (untreated skin) of Rabbit 01 on Day 28.

FIG. 5 is a Table summarizing the results of testing and evaluating Topiramate with six topical formulations on the White New Zealand rabbit model, green background symbolizes 15%+ advantage of the study group over the control, green text over yellow background symbolizes an advantage of 0-15% of the study group over the control, red text over yellow background symbolizes a disadvantage of 0-15% of the study group in comparison to the control, and Red background symbolizes a disadvantage of −15% or more of the study group in comparison to the control.

FIGS. 6a-i are images illustrating the results obtained with aqueous cream (FIGS. 6a, d and g), Nano-emulsion (FIGS. 6 b, e, and h) and Control (FIGS. 6 c, f and i).

FIG. 7 illustrates the wounding performed on the ventral side of the left ear of Rabbit 201; 6 wounds: 3 of 10 mm diameter (upper-middle wound, lower-left wound and lower-right wound) and 3 of 12 mm diameter (upper-left wound, upper-right wound and lower-middle wound).

FIG. 8 illustrates markings of scars (black and blue) and reference skin (blue) in rabbit 205—Left ventral ear on Day 28.

FIG. 9 is a scar histology slide of wound R02 of Rabbit 205 at day 28 of the treatment.

FIG. 10 is a table summarizing the results of the Control and Topiramate aqueous cream treatments (based on Median values). Green background symbolizes 15%+ advantage of the study group over the control, green text over yellow background symbolizes an advantage of 0-15% of the study group over the control, red text over yellow background symbolizes a disadvantage of 0-15% of the study group in comparison to the control, and red background symbolizes a disadvantage of −15% or more of the study group in comparison to the control.

FIGS. 11a-l are photographs of the ear wounds of the silicone Cream Topiramate formulation (2.5%) study groups at days 8, 12 and 16 (FIGS. 11 a, e and i respectively), the aqueous cream Topiramate formulation (2.5%) study group at days 8, 12 and 16 (FIGS. 11 b, f and j respectively) and the untreated control study group at days 8, 12 and 16 (FIGS. 11 c, d, g, h, k and l respectively).

FIGS. 12a-h are microscope images of scar tissue treated with Topiramate and untreated scar tissue.

FIGS. 13a-b illustrate a 21 day treatment of fresh acne scars with 2.5% topiramate in an aqueous crème carrier. FIG. 13a—prior to treatment; FIGS. 13b—21 days post start of treatment.

FIGS. 14a-b illustrate a 30 day treatment of fresh acne scars with 2.5% topiramate in an aqueous crème carrier. FIG. 14a—prior to treatment; FIGS. 14b—30 days post start of treatment.

FIGS. 15a-b illustrate a 60 day treatment of a single Striae atrophy with 2.5% topical Topiramate in an aqueous crème carrier. FIG. 15a—prior to treatment; FIGS. 15b—60 days post start of treatment.

FIG. 16 illustrates the Striae atrophy of FIGS. 15a-b treatment with topical Topiramate 2.5%, surrounded by untreated Striae atrophies.

FIGS. 17a-d illustrate a 90 day treatment of atrophic post-acne scars with 5.0% topical Topiramate in an aqueous crème carrier. FIG. 17a-b—prior to treatment; FIG. 17c-d—90 days post start of treatment.

FIGS. 18a-d illustrate a 90 day treatment of atrophic post-acne scars with 2.5% topical Topiramate in an aqueous crème carrier. FIG. 18a-b—prior to treatment; FIG. 18c-d—90 days post start of treatment.

FIGS. 19a-b illustrate a 90 day prophylactic treatment of skin aging and atrophy with 5.0% topical Topiramate in an aqueous crème carrier. FIG. 19a—prior to treatment; FIG. 19b—90 days post start of treatment.

FIGS. 20a-b illustrate a 42 day treatment of 10 month old, fresh post cesarean scars with 5.0% topical Topiramate in an aqueous crème carrier. FIG. 20a—prior to treatment; FIG. 20b—42 days post start of treatment.

FIGS. 21a-b illustrate a 42 day treatment of 24 month old, mature post cesarean scars with 5.0% topical Topiramate in an aqueous crème carrier. FIG. 21a—prior to treatment; FIG. 21b—42 days post start of treatment.

FIGS. 22a, 22b, 23a, 23b, 24a and 24b illustrate the effects of a 90 day treatment with topiramate oil-in-water cream on facial wrinkles (a—before; b—after).

FIGS. 25a-b illustrate the effects of a 90 day treatment with topiramate oil-in-water cream on stretch marks (a—before; b—after).

### DESCRIPTION OF THE PREFERRED EMBODIMENTS

The present invention is of GABA agonist topical formulations which can be used to treat epithelial disorders such as skin wounds, skin scars and skin atrophy. Specifically, the present invention is of topical Topiramate formulations which are capable of reducing the healing time of fresh wounds, injuries and incisions and concurrently reduce subsequent atrophic and hypertrophic scarring, as well as improve scar appearance and scar tissue quality and reduce scar area, scar length, and scar height above normal uninjured skin. In addition, the formulations of the present invention are capable of improving the state and appearance of a variety of skin disorders including, but not limited to, depressed scarring, widened scarring, flat scarring, irregular scarring, fresh dermatological atrophic scarring, mature dermatological atrophic scarring, fresh surgical atrophic scarring, mature surgical atrophic scarring, congenital atrophic dermatological diseases, acquired atrophic dermatological diseases, acute atrophic dermatological diseases, skin barrier diseases, steroids-induced skin atrophy and Striae, and, skin-aging related disorders.

The principles and operation of the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Although balanced scar formation and remodeling are essential processes in skin wound healing, wounds, scars, skin bather disorders and skin atrophies remain a common and therapeutically refractory clinical problem.

While reducing the present invention to practice, the present inventors have uncovered that Topiramate, a widely used oral anticonvulsant drug is effective in wound, scar, skin barrier and skin atrophy treatment especially when formulated for topical delivery in an oil-in-water carrier.

As used herein, the phrase “epithelial disorders” refers to any disorder that interrupts or causes abnormal growth in epithelial-lined tissue. Examples of disorders include cuts, scratches, wounds, incisional wounds, excisional wounds, sutured wounds, glued wounds, burns, atrophic scars, depressed scars, flat scars, irregular scars, hypertrophic scars, keloid scars, congenital skin atrophy, acute skin atrophy, chronic skin diseases, inflammatory skin diseases, skin barrier disorders, steroids-derived skin atrophy and striae. Examples of epithelium-lined tissue include skin, cornea, lining of organs and the like.

As used herein, the phrase “GABA-agonist” refers to any molecule which can stimulate or increase the action at a GABA receptor, specifically a peripheral GABA receptor present in epithelium-lined tissues. The preferred GABA-agonist of the present invention is Topiramate.

The present formulations were developed with the following specific requirements:

(i) Epithelial penetration and specifically keratinized epithelium penetration (e.g. skin penetration and more specifically, epidermis penetration);
(ii) Restricting effect to local tissue, i.e. tissue under the epithelial layer targeted by the topical formulation of the present invention, e.g. in skin, the dermis or the hypodermis;
(iii) Quick skin absorption with minimal residues;
(iv) Moisturizing effect to overcome skin dryness as a result of treatment;
(v) Easy and rapid application;
(vi) Localizing dosing;
(vii) Minimal local toxicity and sensitization; and
(viii) Stability and long shelf life.

Although topical Topiramate formulations are mentioned in the prior art (U.S. Pat. Appl. No. 20080021094 and U.S. Pat. No. 5,760,006), none of the topical formulations suggested were specifically designed with these parameters in mind or tested as to their efficacy in topical treatment of wounds, scars and skin atrophies. In addition, these prior art references teach that oral formulations and oral doses are preferred in treatment of skin disorders (oral tablets are noted as the preferred approach for psoriasis therapy in U.S. Pat. No. 5,760,006). Oral doses as well as ointment-based topical formulations of Topiramate (WO/2003/097038) are preferred in the prior art since Topiramate is unstable in aqueous environments and thus must be formulated as a dry composition (e.g. tablets) or as a pure oily/fatty composition.

Example 1 of the Examples section which follows describes the different formulations used in the present study as well as provides approaches which can be utilized to manufacture such formulations (see Table 2).

As is further described in the Examples section which follows, several different oil-in-water based formulations having different concentrations of Topiramate and different carrier types were tested for wound healing, post-wounding scar reduction (in area, length and height), fresh Dermatological scar improvement, mature Dermatological scar improvement, fresh surgical scar improvement, mature surgical scar improvement, skin atrophy improvement and skin aging improvement.

The formulations included 0.5% or 2.0% or 5.0% of Topiramate incorporated in a cream, ointment, emulsion or gel bases.

Each one of the specific oil-in-water based formulations tested produced results which were better than placebo or untreated controls in one or more of the parameters tested, however, the present study surprisingly uncovered that some carriers are more suited for topical delivery of Topiramate in the combined treatment of wound healing and the reduction of resulting scarring, and that the effective dose can be substantially lower than that suggested in the prior art (WO/2003/097038).

In scar reduction, all 5 formulation tested outperformed the untreated controls while the oil-in-water formulation [Formulation 1 (aqueous Cream) and Formulation 2 (silicone cream)] produced the best clinical aesthetic results in as far as the combination of wound healing and resulting scar reduction when compared with the control groups. Additional testing also demonstrated that creams and gels were easier to apply, were absorbed quicker than ointment and emulsion formulations, and were stable over extended periods of time when stored at room temperature.

In addition, the aqueous cream formulations were better absorbed than the silicone cream formulations and thus would be more suitable for treatment of fresh and mature scars (older than 7-21 days post skin interruption), whereas in the treatment of fresh scratches, cuts, wounds, fresh trauma wounds, fresh incisional wounds, and fresh excisional wounds (immediately following injury/incision) silicone cream formulations outperformed the aqueous cream formulations possibly due to augmenting Topiramate's wound healing properties by providing a better shielding layer over the wound during its healing process, in an equivalent manner provided by inert silicone-based products such as Dermatix™ or similar products.

In addition to the above, the present studies also uncovered that topical Topiramate concentrations 0.5%-5.0% (w/w) are effective (in comparison to untreated controls and placebo) in both wound therapy and scar therapy and that local topical doses result in systemic doses far below those suggested in the prior art or utilized by prior art oral dose effective treatment regimens.

The present study was conducted in efforts of identifying the formulation most suited for both wound healing and scar prevention/reduction. It was surprisingly uncovered that some formulations were effective in the combination of wound treatment and resulting scar reduction, while others were more effective in scar reduction.

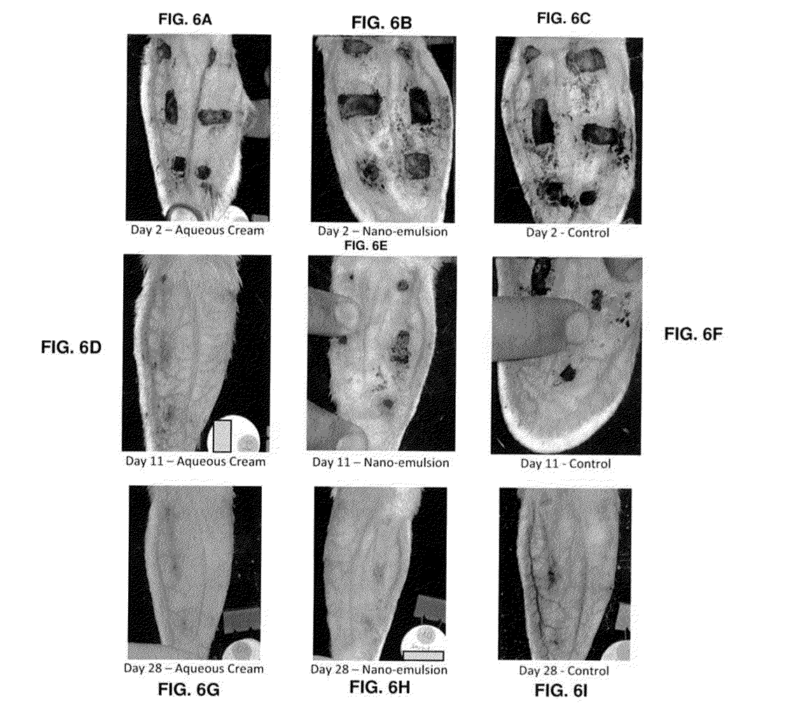

All the formulations tested demonstrated an advantage over the control in the parameters relating to scar reduction/elimination in an excisional wound/scar model. However, surprisingly, only the two oil-in-water cream formulations demonstrated an advantage also in wound healing, while the other formulations were either equal or inferior to control treatment (see FIG. 6).

Formulations based on an oil-in-water cream carrier were further tested on uninterrupted skin barrier disorders (e.g. skin without open wounds or interrupted skin barrier) such as fresh and mature acne scars, post-steroids skin atrophy and striae, fresh and mature cesarean section scars, and as prophylactic treatment for skin aging. As is illustrated in Examples 5-11, oil-in-water cream Topiramate formulations are effective in treatment of aging skin, skin atrophies and atrophic scarring, both fresh and mature, whether such scarring results from a surgical incision, a dermatological disease, a skin disorder or drug use.

Thus, of the present formulations, aqueous cream, silicone cream and gel cream, are most suited for obtaining optimal clinical and aesthetic outcomes in accelerated wound healing, fresh and mature scarring, atrophic skin disorders, autoimmune skin disorders associated with interrupted skin barrier, inflammatory skin disorders associated with interrupted skin barrier and Striae.

Thus, according to one aspect, the present invention provides topical GABA agonist formulations suitable for treatment of wounds, scars, skin disorders resulting in interrupted skin barrier and skin atrophies.

Such a topical GABA agonist formulation is preferably an oil-in-water Topiramate formulation. Such a formulation preferably include 0.1-7.5% Topiramate, more preferably 0.5-5.0% Topiramate formulated in an oil-in-water base which is further described hereinbelow and in the Examples section which follows.

As is clearly demonstrated by the results provided herein, these formulations are highly effective in reducing wound healing time as well as reducing subsequent scarring; and in improving the clinical and aesthetic status of fresh and mature scars skin atrophies, disorders associated with skin barrier interruption/abnormalities and Striae.

Exemplary oil-in-water based formulations include aqueous and silicone based creams such as Formulations 1 and 2 described in the Examples section which follows as well as blue silicone cream, silicone-fluid cream and colloidal hydrous silicate cream (further description of oil-in-water carriers is provided herein below). Oil-in-water formulation can also include gelling agents which may be added to the aqueous phase in order to increase viscosity, such oil-in-water gels also encompass formulations termed herein as gel creams. Such gelling agents can include, water soluble polymers such as sclerotium gum, xanthan gum, sodium alginate, carbomer, cellulose ethers and acrylate polymers which added at 0.5% to 0.75% by weight of the total composition.

Treatment of wounds and resulting scars in the White New Zealand Rabbit Hypertrophic scar model with the oil-in-water aqueous cream formulation of the present invention resulted in an improvement of 72% in the scar elevation index (SEI), a 26% improvement in the scar length index (SLI) and a 78% reduction in scar area when compared to the untreated control. In addition, complete wound epithelization and healing was obtained 18% faster than the control. This formulation was further advantageous in its quick absorption and lack of skin residue following 26 daily applications.

Treatment of wounds and resulting scars in the White New Zealand Rabbit Hypertrophic scar model with the oil-in-water silicone cream formulation of the present invention resulted in a 62% improvement in SEI, a 7.5% improvement in SLI and 47% reduction in scar area when compared to the control. In addition, complete wound epithelization and healing was obtained 14.3% faster than the control. This formulation was also characterized by quick absorption.

In addition to the above, the results provided by the present study demonstrate that treatments started on day 2 post wounding provide superior results in all parameters when compared to treatments started on day 8 post wounding.

These results suggest that in order to optimize wound treatment, one should begin application of the topical formulation of the present invention 0 to 48 hours following injury and continue application until no later than full wound closure. Preferably, treatment with topical Topiramate should start shortly after skin injury, and continue between 7-90 days post injury, depending on the severity of the injury. A single application of a slow release formulation topical Topiramate (such as the oil-in-water nano-emulsion formulation tested), within the window of 0-24 hours following injury can also be beneficial in providing both faster wound healing while reducing and eliminating subsequent scarring in comparison with untreated control.

Thus, the present invention provides topical GABA-agonists formulations and specifically topical Topiramate formulations which are effective in treating wounds, scars, skin disorders associated with skin barrier interruption/abnormalities, skin atrophies and Striae.

In addition to the carriers described herein, the present formulation can also include alternative or additional pharmaceutically acceptable carriers such as, liquid alcohols, liquid glycols, liquid polyalkylene glycols, liquid esters, liquid amides, liquid protein hydrolysates, liquid alkylated protein hydrolysates, liquid lanolin and lanolin derivatives, and like materials commonly employed in cosmetic and medicinal compositions.

Other suitable carriers according to the present invention include, without limitation, alcohols, such as, for example, monohydric and polyhydric alcohols, e.g., ethanol, isopropanol, glycerol, sorbitol, 2-methoxyethanol, diethyleneglycol, ethylene glycol, hexyleneglycol, mannitol, and propylene glycol; ethers such as diethyl or dipropyl ether; polyethylene glycols and methoxypolyoxyethylenes (carbowaxes having molecular weight ranging from 200 to 20,000); polyoxyethylene glycerols, polyoxyethylene sorbitols, stearoyl diacetin, and the like.

Formulations of the present invention can also include a moisturizing agent, for example petrolatum, dimethicone, cyclomethicone, lanoline acid, lanoline alcohol, propylene glycol, cholesterol, cocoa butter and wax. Such moisturizer is of material importance when topical treatment with Topiramate sometimes results in mild-to-moderate feeling of treated skin dryness, which is easily resolved with the application of a moisturizing agent.

Formulations of the present invention can also include a penetration enhancer including, for example, an anionic or cationic surfactant, a fatty acids, a fatty ester, a fatty amine and the like. Such penetration enhancer is of material importance when the formulation of the present invention is used to treat a skin disorder where the skin barrier is intact, such as, but not limited to: fresh scars, mature scars, skin atrophies and Striae.

Additional examples of penetration enhancers suitable for use with the present formulations can be found in “Thong et al. Skin Pharmacol Physiol 2007; 20:272-282”.

The present invention may, if desired, be presented in a dispenser device, such as a tube, a jar, a canister and the like, which may be designed for dispensing one or more unit dosages (either metered or not) containing the topical formulation of the present invention. The dispenser device may be accompanied by instructions for administration, it may also be accompanied by a notice in a form prescribed by a governmental agency regulating the manufacture, use, or sale of pharmaceuticals, which notice is reflective of approval by the agency of the form of the compositions for human or veterinary administration. Such notice, for example, may include labeling approved by the U.S. Food and Drug Administration for prescription drugs or of an approved product insert. Compositions comprising a formulation of the invention may also be prepared, placed in an appropriate container, and labeled for treatment of an indicated condition, as further detailed above.

In addition to the above, Topiramate can also be formulated in alternative carriers in order to treat specific epithelial disorders, such as corneal abrasions.

For example, to treat corneal abrasions, Topiramate can be formulated in a carrier suitable for ophthalmic use.

Suitable ophthalmic carriers are known to those skilled in the art. Carrier types include ophthalmic ointment, cream, gel, gel-cream, foam, solution, or dispersion.

The carrier can also include slow release polymers, stabilizers may also be used (such as, for example, chelating agents, e.g., EDTA), and antioxidants (such as sodium bisulfite, sodium thiosulfite, 8-hydroxy quinoline or ascorbic acid).

Sterility of aqueous formulations can be maintained by conventional ophthalmic preservatives, such as, chlorbutanol, benzalkonium chloride, cetylpyridium chloride, phenyl mercuric salts, thimerosal, and the like; conventional preservatives for ointments include methyl and propyl parabens. In aqueous formulations such agents can be used in amounts which vary from about 0.001 to about 0.1% by weight of the aqueous solution.

Ophthalmic Topiramate formulations may be manually delivered to the eye in suitable dosage form, e.g., eye drops, or delivered by suitable microdrop or spray apparatus typically affording a metered dose of medicament.

Examples of ointment bases include white petrolatum and mineral oil or liquid petrolatum.

Examples of suitable oil-in-water formulations contain minor amounts, i.e., less than about 5% by weight hydroxypropylmethylcellulose, polyvinyl alcohol, carboxymethylcellulose, hydroxyethylcellulose, glycerine and EDTA. The solutions are preferably maintained at substantially neutral pH and isotonic with appropriate amounts of conventional buffers, e.g., phosphate, borate, acetate, tris, etc.

As is mentioned hereinabove, the topical formulations of the present invention can be utilized for treatment of disorders of epithelial lined tissues (referred to herein as epithelial disorders).

Thus, according to another aspect of the present invention, there is provided a method of treating epithelial disorders. The method is effected by topically applying a formulation of the present invention to the affected tissue.

Tables 1a-b below list treatment options using the formulations of the present invention.

TABLE 1a
fresh incisional and excisional wounds

| | Range of Topical | | | |
|---|---|---|---|---|
| | Application (mg. | | | |
| | of topical product | | | |
| Topiramate | application per 100 | Number of | Start (days | |
| Concentration In | square mm. of | applications | after wounding/ | End (days after |
| topical product | wound/incision) | per day | incision) | wounding) |
| 7.00% | 25-150 | 1-2 | 0 | 1 |
| 7.00% | 25-150 | 1-2 | 0 | 4 |
| 7.00% | 25-150 | 1-2 | 1 | 7 |
| 7.00% | 25-150 | 1-2 | 0-8 | Full wound closure |
| | | | | (days 8-21) |
| 5.00% | 25-150 | 1-2 | 0 | 1 |
| 5.00% | 25-150 | 1-2 | 0 | 4 |
| 5.00% | 25-150 | 1-2 | 1 | 7 |
| 5.00% | 25-150 | 1-2 | 0-8 | Full wound closure |
| | | | | (days 8-21) |
| 2.00% | 50-250 | 1-3 | 0 | 1 |
| 2.00% | 50-250 | 1-3 | 0 | 2 |
| 2.00% | 50-250 | 1-3 | 0 | 7 |
| 2.00% | 50-250 | 1-3 | 0-8 | Full wound closure |
| | | | | (days 8-21) |
| 1.00% | 50-250 | 1-3 | 0 | 1 |
| 1.00% | 50-250 | 1-3 | 0 | 2 |
| 1.00% | 50-250 | 1-3 | 0 | 7 |
| 1.00% | 50-250 | 1-3 | 0-8 | Full wound closure |
| | | | | (days 8-21) |

TABLE 1b
mature scars

| | Range of Topical | | | |
|---|---|---|---|---|
| | Application (mg. | | | |
| Topiramate | of topical product | Number of | Treatment | |
| Concentration In | per day per 1 square | applications | Duration | |
| topical product | cm. of scar area) | per day | (days from start) | |
| 7.00% | 100-200 | 1-2 | 45-90 | days |
| 7.00% | 50-150 | 1-2 | 90-180 | days |
| 7.00% | 50-100 | 1-2 | 180-365 | days |
| 5.00% | 100-200 | 1-2 | 45-90 | days |
| 5.00% | 50-150 | 1-2 | 90-180 | days |
| 5.00% | 50-100 | 1-2 | 180-365 | days |
| 2.50% | 200-300 | 1-3 | 45-90 | days |
| 2.50% | 150-250 | 1-3 | 90-180 | days |
| 2.50% | 100-200 | 1-3 | 180-365 | days |
| 1.50% | 200-300 | 1-4 | 45-90 | days |
| 1.50% | 150-250 | 1-4 | 90-180 | days |
| 1.50% | 100-200 | 1-4 | 180-365 | days |

Thus, the present invention also provides methods of treating skin disorders via topical application of a GABA agonist, such as Topiramate.

As used herein, the phrase “topical application” describes application onto a biological surface, whereby the biological surface include, for example, a skin area (e.g., hands, forearms, elbows, legs, face, nails, anus and genital areas as described above) or a mucosal membrane. By selecting the appropriate carrier and optionally other ingredients that can be included in the composition, as is detailed hereinbelow, the compositions of the present invention may be formulated into any form typically employed for topical application. Hence, the compositions of the present invention can be, for example, in a form of a cream, an ointment, a paste, a gel, a gel cream, a lotion, a milk, a suspension, an aerosol, a spray, a foam, a shampoo, a hair conditioner, a serum, a swab, a pledget, a pad, a patch and a soap.

Ointments are semisolid preparations, typically based on petrolatum or petroleum derivatives. The specific ointment base to be used is one that provides for optimum delivery for the active agent chosen for a given formulation, and, preferably, provides for other desired characteristics as well (e.g., emolliency). As with other carriers or vehicles, an ointment base should be inert, stable, nonirritating and nonsensitizing. As explained in Remington: The Science and Practice of Pharmacy, 19th Ed., Easton, Pa.: Mack Publishing Co. (1995), pp. 1399-1404, ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophilic petrolatum.

Emulsion ointment bases are either water-in-oil (W/O) emulsions or oil-in-water (O/W) emulsions, and include, for example, cetyl alcohol, glyceryl monostearate, lanolin and stearic acid. Preferred water-soluble ointment bases are prepared from polyethylene glycols of varying molecular weight.

Lotions are preparations that are to be applied to the skin surface without friction. Lotions are typically liquid or semi-liquid preparations in which solid particles, including the active agent, are present in a water or alcohol base. Lotions are typically preferred for treating large body areas, due to the ease of applying a more fluid composition. Lotions are typically suspensions of solids, and oftentimes comprise a liquid oily emulsion of the oil-in-water type. It is generally necessary that the insoluble matter in a lotion be finely divided. Lotions typically contain suspending agents to produce better dispersions as well as compounds useful for localizing and holding the active agent in contact with the skin, such as methylcellulose, sodium carboxymethylcellulose, and the like.

Creams are viscous liquids or semisolid emulsions, either oil-in-water or water-in-oil. Cream bases are typically water-washable, and contain an oil phase, an emulsifier and an aqueous phase. The oil phase, also called the “internal” phase, is generally comprised of petrolatum and/or a fatty alcohol such as cetyl or stearyl alcohol. The aqueous phase typically, although not necessarily, exceeds the oil phase in volume, and generally contains a humectant. The emulsifier in a cream formulation is generally a nonionic, anionic, cationic or amphoteric surfactant. Reference may be made to Remington: The Science and Practice of Pharmacy, supra, for further information. Pastes are semisolid dosage forms in which the bioactive agent is suspended in a suitable base. Depending on the nature of the base, pastes are divided between fatty pastes or those made from a single-phase aqueous gels. The base in a fatty paste is generally petrolatum, hydrophilic petrolatum and the like. The pastes made from single-phase aqueous gels generally incorporate carboxymethylcellulose or the like as a base. Additional reference may be made to Remington: The Science and Practice of Pharmacy, for further information.

Gel formulations are semisolid, suspension-type systems. Single-phase gels contain organic macromolecules distributed substantially uniformly throughout the carrier liquid, which is typically aqueous, but also, preferably, contain an alcohol and, optionally, an oil. Preferred organic macromolecules, i.e., gelling agents, are crosslinked acrylic acid polymers such as the family of carbomer polymers, e.g., carboxypolyalkylenes that may be obtained commercially under the trademark Carbopol™. Other types of preferred polymers in this context are hydrophilic polymers such as polyethylene oxides, polyoxyethylene-polyoxypropylene copolymers and polyvinylalcohol; cellulosic polymers such as hydroxypropyl cellulose, hydroxyethyl cellulose, hydroxypropyl methylcellulose, hydroxypropyl methylcellulose phthalate, and methyl cellulose; gums such as tragacanth and xanthan gum; sodium alginate; and gelatin. In order to prepare a uniform gel, dispersing agents such as alcohol or glycerin can be added, or the gelling agent can be dispersed by trituration, mechanical mixing or stirring, or combinations thereof.

Sprays generally provide the active agent in an aqueous and/or alcoholic solution which can be misted onto the skin for delivery. Such sprays include those formulated to provide for concentration of the active agent solution at the site of administration following delivery, e.g., the spray solution can be primarily composed of alcohol or other like volatile liquid in which the active agent can be dissolved. Upon delivery to the skin, the carrier evaporates, leaving concentrated active agent at the site of administration.

Foam compositions are typically formulated in a single or multiple phase liquid form and housed in a suitable container, optionally together with a propellant which facilitates the expulsion of the composition from the container, thus transforming it into a foam upon application. Other foam forming techniques include, for example the “Bag-in-a-can” formulation technique. Compositions thus formulated typically contain a low-boiling hydrocarbon, e.g., isopropane. Application and agitation of such a composition at the body temperature cause the isopropane to vaporize and generate the foam, in a manner similar to a pressurized aerosol foaming system. Foams can be water-based or hydroalcoholic, but are frequently formulated with high alcohol content which, upon application to the skin of a user, quickly evaporates, driving the active ingredient through the upper skin layers to the site of treatment.

Skin patches typically comprise a backing, to which a reservoir containing the active agent is attached. The reservoir can be, for example, a pad in which the active agent or composition is dispersed or soaked, or a liquid reservoir. Patches typically further include a frontal water permeable adhesive, which adheres and secures the device to the treated region. Silicone rubbers with self-adhesiveness can alternatively be used. In both cases, a protective permeable layer can be used to protect the adhesive side of the patch prior to its use. Skin patches may further comprise a removable cover, which serves for protecting it upon storage.

Table 1c below provides typical treatment regimen for fresh and mature wounds, fresh and mature scars and various skin disorders using topical formulations of varying topiramate concentrations.

TABLE 1c

| | | Topiramate | Topiramate | Minimum | Minimum | Maximum |
|---|---|---|---|---|---|---|
| | Indication(s)/ | Minimum | Maximum | Treatment | Treatment | Treatment |
| Use Type | Applications | Concentration | Concentration | Frequency | Duration | Duration |
| Acute | Open/fresh | 0.50% | 5.0% | At least | 1 Day | 28 Days |
| Treatment | wounds (non- | | | once a day | | |
| | chronic), cuts, | | | | | |
| | scratches | | | | | |
| | Peeled/ | 0.50% | 5.0% | At least | 1 Day | 90 Days |
| | ablated skin | | | once a day | | |
| | (post chemical | | | | | |
| | peeling, laser, | | | | | |
| | RF, plasma, | | | | | |
| | mechanical | | | | | |
| | dermabrasion | | | | | |
| | treatment) | | | | | |
| | Fresh surgical | 0.50% | 5.0% | At least | 5 Days | 26 weeks |
| | incisions | | | once a day | | |
| | Fresh | 1.0% | 7.5% | At least | 2 weeks | 26 weeks |
| | depressed scars | | | once a day | | |
| | Mature | 1.0% | 7.5% | At least | 4 weeks | 52 weeks |
| | depressed scars | | | once a day | | |
| | Fresh Atrophic | 1.0% | 7.5% | At least | 2 weeks | 26 weeks |
| | Dermatological | | | once a day | | |
| | scars | | | | | |
| | Mature | 1.0% | 7.5% | At least | 4 weeks | 52 weeks |
| | Atrophic | | | once a day | | |
| | Dermatological | | | | | |
| | scars | | | | | |
| | Fresh Atrophic | 1.0% | 7.5% | At least | 2 weeks | 26 weeks |
| | Surgical scars | | | once a day | | |
| | Mature | 1.0% | 7.5% | At least | 4 weeks | 52 weeks |
| | Atrophic | | | once a day | | |
| | Surgical scars | | | | | |
| | Fresh Atrophic | 1.0% | 7.5% | At least | 2 weeks | 26 weeks |
| | Trauma scars | | | once a day | | |
| | Mature | 1.0% | 7.5% | At least | 4 weeks | 52 weeks |
| | Atrophic | | | once a day | | |
| | Trauma scars | | | | | |
| | Striae | 1.0% | 7.5% | At least | 2 weeks | 26 weeks |
| | | | | once a day | | |
| Chronic | Atrophic | 0.1% | 7.5% | At least | 1 week | Lifetime |
| Treatment | congenital skin | | | once a | | use |
| | disorders | | | week | | |
| | Atrophic skin | 0.1% | 7.5% | At least | 1 week | Lifetime |
| | disorders | | | once a | | use |
| | | | | week | | |
| | Skin barrier/ | 0.1% | 7.5% | At least | 1 week | Lifetime |
| | Inflammatory | | | once a | | use |
| | skin diseases | | | week | | |
| | Atrophic/ | 0.1% | 5.0% | At least | | Lifetime |
| | aging skin | | | once a | | use |
| | symptoms: | | | week from | | |
| | wrinkles, | | | the age of | | |
| | cellulite, | | | 30 | | |
| | stretch marks, | | | | | |
| | fine lines, skin | | | | | |
| | sagging, etc. | | | | | |
| Prophylactic | Atrophic/ | 0.01% | 2.5% | At least | | Lifetime |
| Treatment | aging skin | | | once a | | use |
| | symptoms: | | | week from | | |
| | wrinkles, | | | the age of | | |
| | cellulite, | | | 18 | | |
| | stretch marks, | | | | | |
| | fine lines, skin | | | | | |
| | sagging, etc. | | | | | |

As used herein the term “about” refers to ±10%.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

### Example 1

### Selection of Topical Vehicles for Formulation Development

This study aimed to find the appropriate cream, ointment, emulsion or gel base for the purpose of delivering Topiramate topically. A physicochemical characterization of Topiramate using in-silico tools was first conducted in order to select a list of appropriate formulation bases. The physicochemical characterization uncovered that the estimated log P of Topiramate is 1.3 and that its aqueous solubility was approximately 10 mg/ml. In addition, characterization data showed that this drug has some water solubility and also potential affinity to lipids.

Carriers for use in topical semisolid formulations can be classified in terms of their physicochemical properties into four main types:

Fatty bases are anhydrous, they are not absorbed but exert an occlusive effect. They have low capacity to absorb water and are usually used as emollients or as inert vehicles.

Absorption bases are often anhydrous and, typically, consist of a hydrophobic fatty basis in which a water-in-oil emulsifier has been incorporated. Bases of this type are used as vehicles for aqueous liquids or solution of medicaments. They are not easily removed from the skin.

Emulsion bases are absorption bases to which water has been added to give water-in-oil or oil-in-water emulsions. The water-in-oil bases have occlusive properties and because of their oily external phase are less readily removed by water. Oil-in-water variety are the most cosmetically elegant, they are easily spread on the skin and readily form vanishing-type creams on admixture with water.

Water soluble bases—most water-soluble ointment bases are made by blending macrogols of high and low molecular weight.

From a pharmaceutical perspective, Topiramate is suitable for incorporation into oil-in-water based formulations such as creams, gel creams, gel (alcoholic and non-alcoholic), foam (water-based and hydroalcoholic), emulsions, nano-emulsions and lotions due to its partial aqueous solubility, and, also into fatty ointments due to its hydrophobic properties potential affinity to lipids, and, into alcoholic and non-alcoholic based foam formulations.

Thus, oil-in-water bases, hydroalcoholic bases and fatty ointments were selected and evaluated for animal studies.

The proposed formulations included the following carriers:
1. Aqueous cream (BP): water, white soft paraffin, cetosteary alcohol, liquid paraffin and sodium lauryl sulfate.
2. Cetomacrogol cream (British pharmaceutical codex): water, white soft paraffin, cetosteary alcohol, liquid paraffin and cetomacrogol 1000.
3. Blue silicon cream: water, dimethicone, stearic acid, isopropyl myristate, mineral oil, glycerin, glyceril stearate, cetyl alcohol, pentenol and TEA.
4. Hydrous wool fat (lanolin) which contains wool fat and water and is water-in-oil emulsion base.
5. Emulsifying ointment BP which contains soft paraffin, liquid paraffin, cetosterayl alcohol and SLS. This base is oil-in-water emulsion base.
6. Cetomacrogol emulsifying ointment BP containing soft paraffin, liquid paraffin, cetostearyl alcohol and cetomacrogol 1000. This base is also oil-in-water base.
7. Oil-in-water Nanoemulsions containing 5-20% oil phase and 80-95 water phase emulsified using phospholipids and non-ionic surfactants.
8. Topical transparent aqueous gels based on Carbopol polycarboxilic polymers.
9. Hydroalcoholic foam formulations containing 60% of ethanol for topical applications. The advantages are quick evaporation and skin penetration due high alcohol content.
9. Water-based foam formulations containing 60% of water for topical application. The advantages are lower probability of irritation without leaving any greasy residues. Administration of the foam using a metered-dose dispensing device will provide a measured dose of the drug.

Table 2 below describes the various base (carrier) formulations and method of manufacturing.

TABLE 2

| | Composition | Procedure | Classification |
|---|---|---|---|
| Cetomacrogol | Cetomacrogol 1000- 200 g | Melt together and stir until | |
| emulsifying wax | Cetostearyl alcohol- 800 g | cold | |
| Cetomacrogol | Cetomacrogol emulsifying wax- 300 g | Melt together and stir until | Emulsion base. Oil in |
| emulsifying oint | Liquid paraffin- 200 g | cold | water (nonionic) |
| | White soft paraffin- 500 g | | |
| Cetomacrogol cream A | Cetomacrogol emulsifying oint- 300 g | Dissolve the preservatives in | Oil in water creams |
| | Chlorocresol- 1 g | water with the aid of gentle | which are stable over a |
| | Water- 699 g | heat. Melt the oint on a water | wide pH range and |
| Cetomacrogol cream B | Cetomacrogol emulsifying oint- 300 g | bath, add the solution | suitable for the |
| | Propyl hydroxybenzoate- 0.8 g | containing the preservative at | incorporation of many |
| | Methyl hydroxybenzoate- 1.5 g | the same temp and stir until | anioninc, cationic and |
| | Benzyl alcohol- 15 g | cold. | non-ionic medicaments |
| | Water - 682.7 g | Improved product may | |
| | | obtained if instead of using | |
| | | the oint, the appropriate | |
| | | quantities of Cetomacrogol | |
| | | emulsifying wax, Liquid | |
| | | paraffin and | |
| | | White soft paraffin are | |
| | | melted together | |
| Emulsifying wax | Sodium lauryl sulfate- 10 g | Melt the cetostearl to 95° C. | |
| (anionic emulsifying | Cetosteary alcohol- 90 g | add SLS, mix and add the | |
| wax) | Water- 4 g | water heat to 115° C. and | |
| | | maintain at this temp, stir | |
| | | vigorously until ftothing | |
| | | ceases and the product in | |
| | | translucent, cool rapidly | |
| Emulsifying ointment | Emulsifying wax- 300 g | Melt together and stir until | Emulsion base. Oil in |
| | White soft paraffin- 500 g | cold | water (anionic) |
| | Liquid paraffin- 200 g | | |
| Aqueous cream | Emulsifying ointment- 300 g | Dissolve the chlorcresol in | |
| (hydrous emulsifying | Chlorcresol- 1 g | water with the aid of gentle | |
| ointment) | Water - 699 g | heat. Melt the oint, add the | |
| | | chlorcresol solution at the | |
| | | same temp and stir gently | |
| | | until cold | |
| Cetrimide emulsifing | Cetrimide- 5 g | | Emulsion base. Oil in |
| oint | Cetosteary alcohol- 50 g | | water (cationic) |
| | Liquid paraffin- 500 g | | |
| | Water ad 1 kg | | |
| Dimethicon cream | Dimethocone 350-100 g | Warm and mix together the | Don't apply on |
| (silicone cream) | Cetrimide- 5 g | dimethicone, liquid paraffin, | inflamed or broken |
| | Chlorcresol- 1 g | and cetostearyl alcohol until | skin |
| | Cetosteary alcohol- 50 g | homogenous, add with | |
| | Liquid paraffin- 400 g | mechanical stirring the | |
| | Water- 444 g | cetrimide and chlorocresol | |
| | | dissolved in water at the same | |
| | | temp and stir until cold | |
| Macrogol oint | Macrogol 4000- 350 g | Melt macrogol 4000, add the | Water soluble |
| | Macrogol 300- 650 g | macrogol 300 and stir until | basis. suitable for water |
| | | cold | soluble drugs. Have no |
| | | | protective or emollient |
| | | | properties, easily |
| | | | washed from skin |
| | | | surface, non greasy. |
| | | | Reduce antimicrobial |
| | | | potency, dissolve |
| | | | certain plastics |
| | | | (container issue) |
| Paraffin oint | Hard paraffin- 30 g | Melt together and stir until | Fatty base. Not |
| | White soft paraffin (Vaseline)- 900 g | cold | absorbed, occlusive |
| | White beeswax- 20 g | | effect, greasy, difficult |
| | Cetostearyl alcohol- 50 g | | to remove |
| Simple oint | Cetostearyl alcohol- 50 g | Melt together and stir until | |
| | Hard paraffin- 50 g | cold | |
| | Wool fat- 50 g | | |
| | White soft paraffin or yellow | | |
| | paraffin- 850 g | | |
| Wool alcohols oint | Wool alcohols- 60 | Melt together and stir until | |
| | White or yellow soft paraffin- 100 g | cold | |
| | Hard paraffin- 240 g | | |
| | Liquid paraffin- 600 g | | |
| Oily cream | Wool alcohols oint- 500 g | Melt the wool alcohols oint | Emulsion base. Water |
| | Water- 500 g | and add gradually, with | in oil. Occlusive |
| | | constant stirring the water at | properties, less readily |
| | | 50° C. mix vigorously until a | removed by water |
| | | smooth cream is obtained and | |
| | | stir until cold | |
| Hydrous wool fat | Wool fat (anhydrous lanolin)- 700 g | | Emulsion base. Water |
| | Water- 300 ml | | in oil. Occlusive |
| | | | properties, less readily |
| | | | removed by water |
| Hydrous wool fat oint | Hydrous wool fat (lanolin)- 500 g | | |
| | Yellow soft paraffin- 500 g | | |
| Foam - | Ethanol (60%), purified water, | | |
| Hydroalcoholic | propylene glycol, cetyl alcohol, | | |
| | strearyl alcohol, polysorbate 60, | | |
| | citric acid, pottasium citrate | | |
| Foam - water-based | water (60%), mineral oil, | | |
| | isopropyl myristate, MCT oil, | | |
| | glyceryl monostreate, strearyl | | |
| | alcohol, xantan gum, methocel | | |
| | K1000M, TWEEN 80, MYRJ | | |
| | 49p, Glycofurol, | | |
| | cocoamidopropylbethaine, | | |
| | phenonip, butane | | |

Six formulations were selected for further studies based on their suitability for treating topical wounds and resulting scar reduction, as well as for ease and comfort of application and post application.

### Example 2

### Safety and Efficacy of Topical Topiramate Formulations

The purpose of this study was to evaluate the safety and efficacy (wound healing and hypertrophic scar reduction) properties of 6 different oil-in-water based topical formulations of Topiramate in the White New Zealand Rabbit's hypertrophic scar model.

Each formulation containing 5.0% Topiramate was objectively compared to a placebo (no Topiramate) of the same formulation and to an untreated control (no Topiramate) using the following parameters:

(i) SEI (Scar Elevation Index)=ratio between the area of the cross section of the scar, and the cross section of a normal skin in the same dimensions. Any value above 1 means some level of scar elevation above normal uninjured skin level.

(ii) SLI (Scar Length Index)=ratio between the scar longest axis (Day of sampling) and the wound longest axis at injury (Day 0). Any value above 1 means the scar is longer than the length of the fresh wound generated

(iii) SWR (Scar to Wound surface Ratio)=ratio between the final scar surface area (in sq. mm) at Day of sampling and the wound area in Day 2 (prior to treatment beginning). This parameter enables to evaluate the scar surface area reduction properties of the drug+formulation combination

(iv) TTH (Time to Healing)=average number in days from Day 0 (wounding) to full wound healing. TTH is separately calculated for each individual wound, and per a group of wounds treated in the same protocol.

Materials and Methods

Fourteen White New Zealand Rabbits were tested over a time period of 29 days. 2 rabbits were allocated for treatment by each of the 6 topical formulations, and 2 rabbits were allocated to the untreated control group.

Six different topical formulations containing 5% of Topiramate (w/w) were developed and optimized for this study (Aminolab Pharma, Ness Tziona, Israel):

Formulation 1—Aqueous cream (BP) consisting of water, white soft paraffin, cetosteary alcohol, liquid paraffin and sodium lauryl sulfate.

Formulation 2—Silicon cream consisting of water, dimethicone, stearic acid, isopropyl myristate, mineral oil, glycerin, glyceryl stearate, cetyl alcohol, pentenol and TEA.

Formulation 3—Emulsifying ointment BP consisting of soft paraffin, liquid paraffin, cetosterayl alcohol and SLS. This base is oil-in-water emulsion base.

Formulation 4—Cetomacrogol emulsifying ointment BP consisting of soft paraffin, liquid paraffin, cetostearyl alcohol and cetomacrogol 1000. This carrier is an oil-in-water base.

Formulation 5—Oil-in-water Nanoemulsion consisting of 5-20% oil phase and 80-95 water phase emulsified using phospholipids and non-ionic surfactants.

Formulation 6—Topical transparent aqueous gel based on Carbopol polycarboxilic polymers.

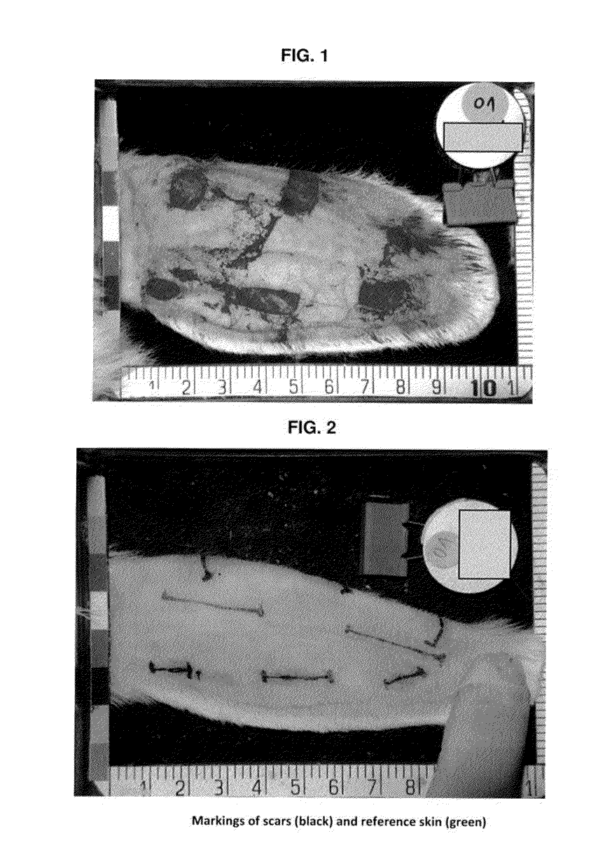

On Day 0, each rabbit was anesthetized, and 6 full thickness wounds exposing the cartilage were generated on the ventral side of each ear (see FIG. 1). The wounds were similar in size and shape in all rabbits.

Each wound received a unique enumerator, used for individual wound identification and follow-up. On the right ear, wounds were enumerated R01-R06, and on the left Ear L01-L06.

On day 2 of the study (e.g. between 36-48 hours post wounding), each rabbit of the six study groups started treatment with each specific 5% Topiramate topical formulation. Six wounds on one ear were treated with a specific formulation, while on the opposite ear, 3 wounds were treated with a specific Placebo formulation (i.e. identical to the formulation used in the first ear minus Topiramate). On day 8 of the study, the remaining 3 wounds not treated until this stage, started treatment with the 5% Topiramate formulations. This means, that in each rabbit, 6 wounds were treated with the specific topical Topiramate from day 2 until the end of the study, 3 wounds were treated with the specific topical Topiramate from day 2 until the end of the study, and; 3 wounds were treated by the placebo version of the specific formulation from day 2 until the end of the study.

The weight of the Topiramate and Placebo containers was determined using a portable highly sensitive weight (with a 10 mg accuracy capability) prior to, and following each application to each individual ear, so as to enable very accurate measurements of daily and aggregate Topiramate applied (Tables 3a-b).

TABLE 3a
Daily application of Topiramate per wound

| | Average Topiramate | Average Topiramate |
|---|---|---|
| | Application per | Application per |
| | Wound treated between | Wound treated between |
| Treatment | D 2-D 28 (in mg.) | D 8-D 28 (in mg.) |
| Aqueous Cream | 5.84 | 6.07 |
| Silicone Cream | 5.62 | 5.56 |
| Emulsifying | 5.38 | 4.71 |
| Ointment | | |
| Ceto. Ointment | 5.23 | 5.24 |
| Nano-emulsion | 6.46 | 6.16 |

TABLE 3b
Daily application of Topiramate per 100 mm² of excisional wound

| | Average Topiramate | Average Topiramate |
|---|---|---|
| | Daily Application per | Daily Application per |
| | 100 square mm. of | 100 square mm. of |
| | excisional wound | excisional wound |
| | treated between | treated between |
| Treatment | D 2-D 28 (in mg.) | D 8-D 28 (in mg.) |
| Aqueous Cream | 5.17 | 5.38 |
| Silicone Cream | 4.98 | 4.93 |
| Emulsifying | 4.76 | 4.18 |
| Ointment | | |
| Ceto. Ointment | 4.63 | 4.65 |
| Nano-emulsion | 5.72 | 5.46 |

In addition, each ear and wound of each rabbit, was photographed using a high resolution 12 Megapixel camera (G9, Canon, Japan) and a special purpose dermatology to photography/scaling/measurement apparatus (FotoFinder, Bad Birnbach, Germany). Photographs were taken on days 0, 2, 5, 9, 11, 13, 15, 17, 19 and 28, enabling accurate 0.1 mm resolution measurement of wound length, wound area, scar area and scar axis length.

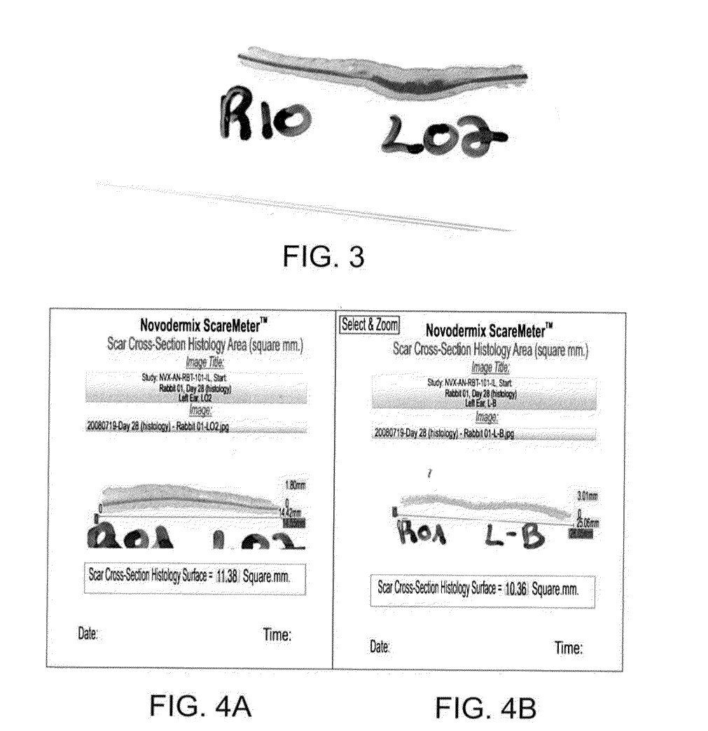

On day 29, all rabbits were sacrificed, and the longest axis of each scar (resulting from wounding on day 0) was marked over the scar using a standard non-erasable Black marker (see FIG. 2 below). In addition to the marking of each scar, 2 uninjured and untreated skin sites, averaging 25 mm each were also marked (in Green color). After marking, a cross-sectional histological sample was taken from each wound and reference exactly according to the marking, placed in formaldehyde and sent for histological slide preparation (see FIG. 3). Based on the histological example length, and the photographed histological slides, the area of the scar (above cartilage) and the area of the adjacent normal skin were calculated and recorded (see FIG. 4).

Results

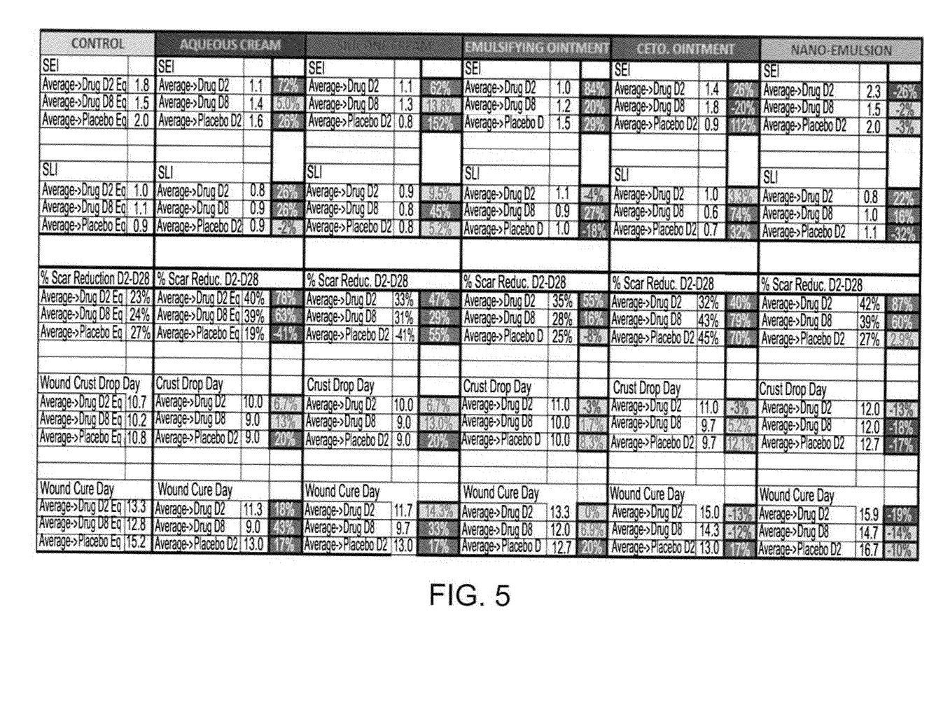

The results of the Control group and five of the study Groups is presented in FIG. 5. Formulation 1 (aqueous Cream) and Formulation 2 (silicone cream) produced the best results in as far as combined wound healing and scar reduction/elimination in comparison with the untreated control group.

The Aqueous Cream formulation treatment resulted in 72% better SEI, 26% better SLI and 78% smaller scar area in comparison to the untreated control. At the same time, complete wound healing was obtained 18% earlier as compared with the control. This formulation was further advantageous in its quick absorption and lack of skin residue following continuous 26 days application.

The silicone cream formulation treatment resulted in 62% better SEI, 7.5% better SLI and 47% smaller scar area in comparison to the control. At the same time, complete wound healing was obtained 14.3% earlier as compared with the control. This formulation was also characterized by quick absorption, yet was rated slightly lower than Aqueous Cream due to its silicone residue.

The Emulsifying ointment formulation treatment resulted in 84% better SEI, and 55% smaller scar area in comparison to the control. However, this formulation was 4% lower than the control in as far as SLI, and was just equal to the control in its wound healing. This formulation was rated relatively low in terms of user skin application experience, slow absorption, and left a greasy residue over the wound.

The Ceto Ointment formulation treatment resulted in 26% better SEI, 3.3% better SLI and 40% smaller scar area in comparison to the control. At the same time, complete wound healing was obtained 18% earlier as compared with the control. However, this formulation was 13% lower than the control in its wound healing properties. This formulation was also rated relatively low in terms of user skin application experience, slow absorption, and left a greasy residue over the wound.

With regards to the Gel formulation, in the several observations made during the beginning of the study, the Gel formulation had a negative impact on the wound healing process, similar to the negative effects of the Nano-emulsion formulation.

The Nano-emulsion formulation treatment resulted in 22% better SLI and 87% smaller scar area in comparison to the control. However, this formulation was 26% lower than the control in its SEI and 19% lower in its wound healing properties in comparison with the control. This formulation was further rated high in its very pleasant feel and quick absorption and by leaving no residues on the skin.

Furthermore, comparison of treatments started on day 2 and identical treatments started on day 8 demonstrated an advantage in all parameters to the treatment started on day 2.

In conclusion, all the oil-in-water formulations tested demonstrated an advantage over the control in the parameters relating to scar reduction/elimination in an excisional wound/scar model. However, surprisingly, only the two cream formulations demonstrated an advantage also in wound healing properties, while the other formulations were either equal or inferior to control treatment (see FIG. 6).

### Example 3

### Safety and Efficacy of Formulations 1 and 2

The purpose of this study was to evaluate the safety and efficacy (wound healing and scar reduction) properties of 2 concentrations of Topiramate (2.0% and 0.5%) of the 2 best performing oil-in-water formulations: Aqueous Cream (formulation 1) and Silicone Cream (formulation 2) described in Example 2. Comparison of each formulation vs. the control was effected according to the parameters described in Example 2.

Materials and Methods

Six White New Zealand Rabbits were treated over a time period of 29 days. Two rabbits were allocated for treatment by each of the 2 topical formulations (formulations 1 and 2 described in Example 2), and 2 rabbits were allocated to the control group (no treatment at all). Each of the 2 formulation included 2 different concentrations of Topiramate, 2.0% and 0.5%.

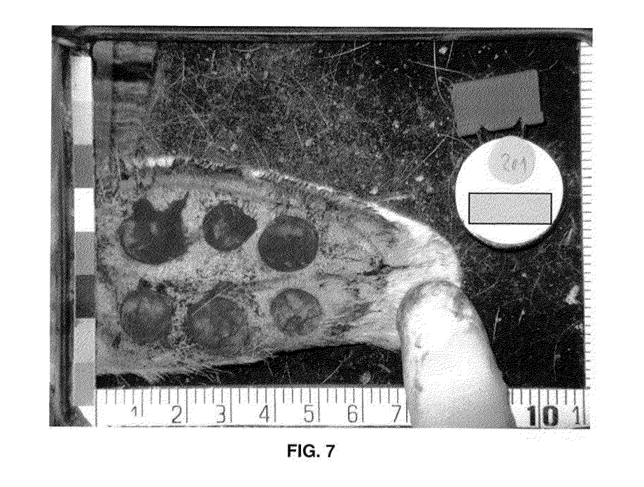

On Day 0, each rabbit was anesthetized, and 6 full thickness excisional wounds were generated on the ventral side of each ear using a punch biopsy (Acuderm, USA) fully exposing the cartilage. Three of the six excisional wounds generated in each ear were 10 mm in diameter, and the other 3 wounds were 12 mm in diameter. All wounds were identical in size and shape in all rabbits (see FIG. 7).

Each wound received a unique enumerator, used for individual wound follow-up. On the right ear, wounds were designated R01-R06, and on the left ear L01-L06.

At day 2 of the study, treatment with 2.0% Topiramate formulation was initiated on 6 wounds on one ear of each of the 2 rabbits in the two study groups; while the opposite ears of the same rabbits were treated with a 0.5% Topiramate formulation.

The Topiramate and placebo containers were weighed as described above prior to, and following application, so as to enable assessment of daily and aggregate Topiramate application. Table 4 below provides the average daily application of Topiramate, in milligrams per wound in each of the four treatments.

TABLE 4
Daily application, per wound, of topical Topiramate

| | | Average Topiramate |
|---|---|---|
| | Average Topiramate | Application per |
| | Application | each 100 square |
| | per Wound per | mm. of excisional wound |
| Treatment | day (in mg.) | area per day (in mg.) |
| Aqueous Cream 2.5% | 4.85 | 5.06 |
| Aqueous Cream 0.5% | 0.99 | 1.03 |
| Silicone Cream 2.5% | 4.49 | 4.69 |
| Silicone Cream 0.5% | 0.95 | 0.99 |

In addition, each ear of each rabbit was photographed as described above. Photographs were taken on days 0, 2, 5, 8, 10, 12, 14, 16, 18, 20, 24 and 28, enabling accurate 0.1 mm resolution measurement of wound length, wound area, scar area and scar axis length.

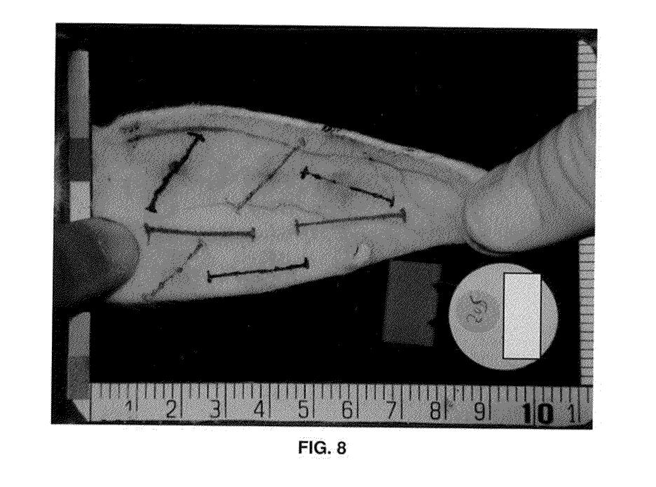

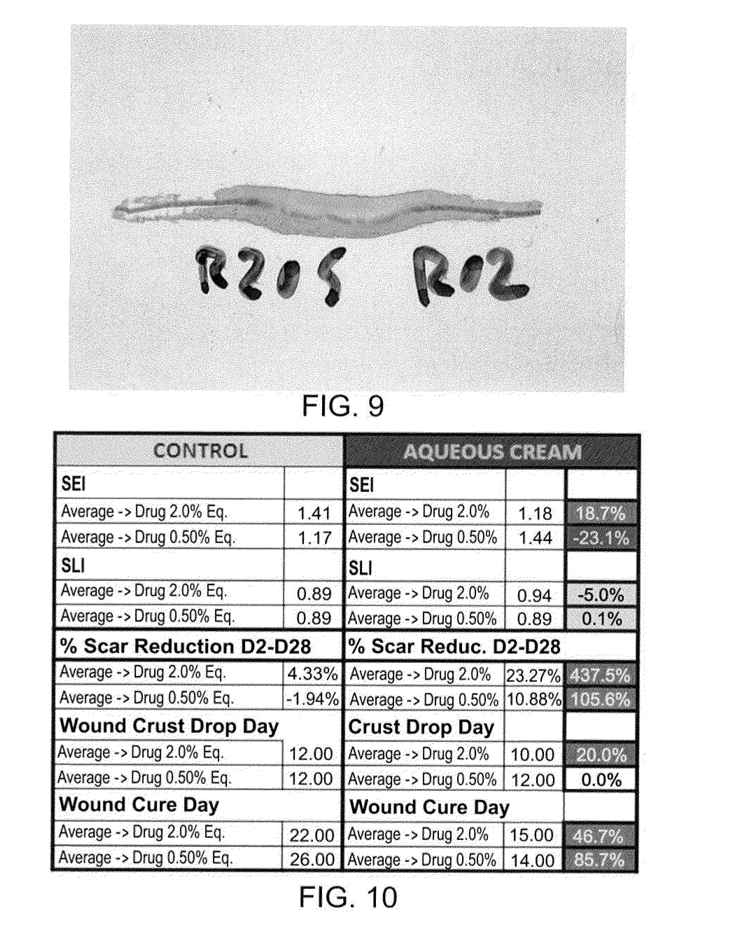

On day 29, all rabbits were sacrificed and the longest axis of each scar (resulting from day 0 wounding) was marked using a standard non-erasable black or blue marker (see FIG. 8). In addition, 2 reference uninjured and untreated skin sites, each approximately 25 mm in diameter, were also marked (in blue color). Following marking, a cross-sectional histological sample was taken from each wound and was referenced to the marking. The sample was placed in formaldehyde and sent for histological slide preparation (see FIG. 9). Based on the histological data and the photographs, the area of the scar (above cartilage) and the area of the adjacent normal skin were calculated and recorded.

Results

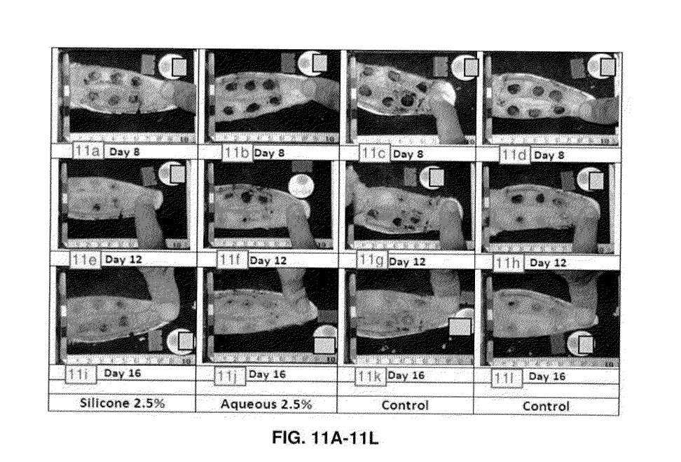

The results of the Control group and the Aqueous Cream Topiramate formulation group is presented in FIG. 10. Although the rabbits in the Silicone Cream treatment group did not finish the entire treatment period, the comparative data presented below (in FIGS. 11a-l), demonstrate that by day 16, the efficacy of the 2.0% Topiramate-silicone cream formulation was similar to the wound healing and scar reduction efficacy of the 2.0% Topiramate-aqueous cream formulation.

More specifically, the 2.0% Topiramate-Silicone Cream formulation, produced better results in wound closure/healing than the 2.0% Topiramate-aqueous cream formulation, and better than the control group, on days 12 and 16. As both rabbits treated with the silicone cream formulation did not complete the study due to premature death unrelated to the study, the other parameters of this group (SEI, SLI, etc.) could not be compared.

The 2.0% Topiramate-aqueous cream formulation treatment started on Day 2 obtained a 18.7% advantage over the control in SEI, a 437.5% advantage in scar area reduction vs. control, a 20% advantages in median wound crust drop day over the control, and a 46.7% advantages over the control in median full wound healing day. However, in the SLI (Scar Length Index) parameter, this treatment group was inferior to the control (by 5%).

The 0.5% Topiramate—aqueous cream formulation treatment started on Day 2 was 23.1% lower than the control in SEI, but obtained a 105.6% advantage in scar area reduction vs. control. This formulation was also equal to the control in wound crust drop day and 85.7% higher than the control in median full wound healing day. In the SLI (Scar Length Index) parameter, this treatment group was also equal to the control group.

In conclusion, both the 2.0% Topiramate-aqueous cream formulation and the 2.0% Topiramate-silicone cream formulation performed better than the Control group. The 0.5% Topiramate-aqueous cream formulation and the 0.5% Topiramate-silicone cream formulation also performed better than the control in some parameters. Overall, this study demonstrated that treatment with a 2.0% Topiramate formulation is superior to treatment with a 0.5% Topiramate formulation.

### Example 4

### Histology of Tissue from Healed Wounds and Scars

Rabbits treated with formulations 1, 2 and 5 and rabbits from the control group in Example 2 were sacrificed and scar tissue was histologically evaluated. The tissue slides were evaluated under an Olympus light microscope at 10-40× magnifications.

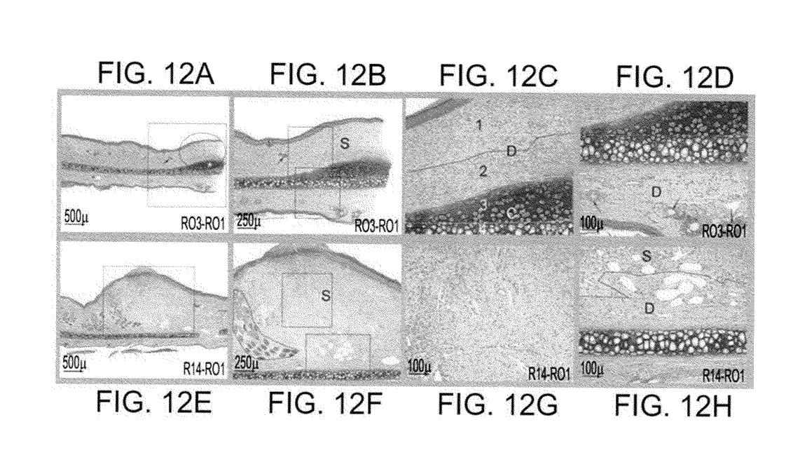

FIGS. 12a-h illustrate images of wound tissue captured from the microscope using an Olympus digital camera. FIG. 12a is a low power view of sample R03-01 treated with the 5.0% Topiramate silicone formulation. The scar is located at the top right edge of the sample, approximately in the circled area. The level of the skin surface is similar to that of the normal skin. Below the fibrous scar there is an area of new cartilage formation (white asterisk). A hair follicle on the left margin of the scar is indicated with an arrow. The boxed area is shown at higher power in FIG. 12b in which hair follicles are absent from the area of the scar (S). An arrow indicates a hair follicle at the edge of the scar. Fibrous tissue continues a short way beyond the hair follicle and blends imperceptibly with normal dermal collagen on the left of the field.

FIG. 12c is a high power magnification of the top boxed area in FIG. 12b. Most or all dermal collagen is replace by fibrous scar tissue in this field. In the area marked as 1, above the black line, extracellular material is more abundant than cells whilst in the area marked as 2 cells (fibroblasts) predominate. Below the fibrous tissue there is an area of new cartilage formation (3, delineated in white) which lies above the original pinnal cartilage (4). D—dermis, C—cartilage.

FIG. 12d is a high power magnification of the bottom boxed area in FIG. 12b showing the histological appearance of normal dermis (D) with thick bundles of collagen and a relatively low number of inconspicuous fibroblasts. Arrows indicate hair follicles.

FIG. 12e is a low power view of sample R14-R01, an untreated control. Scar tissue forms a mound like swelling in the center of the boxed area.

FIG. 12f is a medium power magnification of the boxed area in FIG. 12e. The scar (S) is devoid of hair follicles. An large cluster of hair follicle is located at the lateral margin of the scar (surrounded in black).

FIG. 12g is a high power magnification of the top boxed area in FIG. 12f. The fibrous scar tissue is composed of an admixture of collagen and fibroblasts. The ratio between these two components is similar to that of area 2 in image C. The orientation of the collagen fibers is much less uniform than in C, where they lie parallel to the skin surface.

FIG. 12h is a high power magnification of the bottom boxed area in FIG. 12f. Scar tissue (S) does not extend to the level of the cartilage. A thin layer of normal dermis (D, below black line) is preserved above the cartilage. Note similarity of this tissue to the dermis on the opposite side of the cartilage. Absence of reactive changes in the pinnal cartilage is also related to preservation of the deepest dermis.

Table 5 below summarizes the architecture of the tissue as observed under the light microscope.

TABLE 5
histological findings

| ID | Epidermis | Dermis | other |
|---|---|---|---|
| R01-RB | Wide side: Av: 50μ, | Wide side: mild mf MN infiltration | Note: Inflammation is |
| NAÏVE | Thick: 80μ | and focal crust formation. | present in this control |
| (untreated & | Narrow side: 10-30μ, | Skin (epidermis + dermis) is ~750- | slide. |
| unwounded) | Av: 20μ | 1000μ wide on one side (with a | |
| CONTROL | | large blood vessel) and ~300-400μ | |
| | | on the other - referred to as wide | |
| | | and narrow sides respectively. | |
| R01-R02 | Wide side: Av: 80μ, | Wide side: mf non-delineated | Missing superficial |
| Topiramate, | Thick: 100μ | fibrosis, mild mf NN and H | dermis over half of the |
| aqueous | Narrow side: Av: 25μ | infiltration, Dermal defect not seen. | sample. |
| | (and HK) | | |
| R01-R05 | Wide side: Thick: 220μ | Wide side: extensive but non | A very large follicle in |
| Topiramate, | rest is variable. | contiguous fibrosis with mf loss of | the middle of the scar. |
| aqueous | Widespread crusts | HF, admixed mild mf MN + H | |
| | Narrow side: Av: 25μ | infiltration. | |
| R01-L02 | Wide side: Av: 80μ, | Wide side: mf non-delineated | Similar to R01-02 |
| Placebo, | Thick: 100μ | fibrosis, mild mf NN and H | |
| aqueous | Narrow side: WNL (HK) | infiltration, Dermal defect not seen. | |
| R03-RB | Wide: 30-50μ WNL | WNL | Narrow: ~250μ, |
| NAÏVE | Narrow: 15-25μ WNL | | wide: 500-1000μ. |
| (untreated & | | | |
| unwounded) | | | |
| CONTROL | | | |
| R03-R01 | Wide: 40-50μ | Widespread fibrosis, more in | Cartilage formation at |
| Topiramate, | Narrow: WNL (HK) | deeper dermis, mostly without loss | the edge in the area of |
| silicone | | of HF. Focal loss of HF i.e a | dermal defect. |
| | | dermal defect at the edge. | |
| R03-R02 | | Dermal defect not seen. | Missing all superficial |
| Topiramate, | | | dermis on one side. |
| silicone | | | |
| R03-L02 | Wide: 40-50μ | Locally extensive fibrosis including | |
| Placebo, | Narrow: WNL | a dermal defect - 7 mm wide but | |
| silicone | | with a small HF in its middle. | |
| | | The fibrosis appears relatively | |
| | | mature - as evaluated by | |
| | | 1) lesser swelling/ | |
| | | hypertrophy of fibroblasts | |
| | | (cf. above samples). | |
| | | 2) 2) Sunken surface | |
| R10-LB | “Wide”: 40-50μ | WNL | Minimal difference |
| NAÏVE | “Narrow”: 20-25μ/WNL | | between wide and |
| (untreated & | | | narrow sides - both |
| unwounded) | | | are ~300-350μ. |
| CONTROL | | | Cartilage is |
| | | | discontinuous. |
| R10-L01 | Wide: 70-80μ | Wide: Locally extensive relatively | Cartilage formation in |
| Topiramate, | Narrow: Av: 25μ, | mature fibrosis within which there | the area of fibrosis. |
| nano-emulsion | Thick: 40-50μ | is incomplete loss of HF. Mild mf | |
| | | MN infiltration in the fibrotic area. | |
| | | Edges of fibrosis difficult to define. | |
| | | Narrow: mild fibrosis deep dermis | |
| | | over area of cartilage formation. | |
| R10-L05 | Wide: Thick: 250μ, | Wide: Locally extensive scar and | Pronounced cartilage |
| Topiramate, | Av; 70μ | dermal defect 5 mm long. The scar | formation in the area of |
| nano-emulsion | Narrow: 30-50μ | is swollen/protrudes above | fibrosis. |
| | | flanking tissue and hypercellular | |
| | | due to spindle cells and MN cells | |
| R10-R02 | Wide: Thick 300μ - mark | Wide: Locally extensive scar and | Pronounced cartilage |
| Placebo, nano- | hyperplasia, crusts. | dermal defect 1.3 cm long. | formation in the area of |
| emulsion | Narrow: ~100μ | Feature similar to R10-L05 but less | fibrosis. |
| | | swollen. | |
| | | Narrow: mf fibrosis. | |
| R14-RB | Wide: Av: 25μ, WNL | WNL | No significant difference |
| NAÏVE | Narrow: Av: 15-25μ, | | between sides either in |
| (untreated & | WNL | | dermis or epidermis. |
| unwounded) | | | |
| CONTROL | | | |
| R14-R01 | Wide: Thick: 100μ with | Wide: Locally extensive scar and | |
| NON- | crust Av: ~50μ | dermal defect 3 mm wide forming | |
| TREATED | Narrow: WNL | a dome. Scar extends beyond the | |
| | | slopes of the dome and contains 2 | |
| | | HF. | |
| R14-R05 | Wide: Thick ~100μ, | Wide: A 5 mm wide fibrotic dome | Focal cartilage formation |
| NON- | crusts, Av: 50μ | including HF. Fibrous tissue | in the area of fibrosis. |
| TREATED | Narrow: WNL | relatively mature and extends | |
| | | beyond slopes of dome. The | |
| | | domed contour appears to be at | |
| | | least partly due to the cartilage | |
| | | formation. | |
| R14-L01 | Wide: Thick 250μ, | Wide: Locally extensive scar and | |
| NON- | crusts, Av: 100μ | dermal defect 5 mm wide forming | |
| TREATED | Narrow: WNL | a dome. Fibrous tissue is | |
| | | moderately mature. | |

Explanation of terms
Crusts - i.e. serocellular crusts - areas of purulent exudative inflammation
Dermal defect - a fibrotic area devoid of hair follicles indicating tissue loss.
Non-delineated - patchy, discohesive, difficult to demarcate.
Abbreviations:
Av—average
H—heterophils
HF—hair follicle
HK—hyperkeratosis
Mf: multifocal
MN: mononuclear cells. Includes histiocytes/macrophages, lymphocytes and plasmacells.
WNL—within normal limits

The effect of each tested formulation is described in Table 6 below. These findings clearly illustrate that the aqueous Topiramate formulation (formulation 1) provides the best wound healing results.

TABLE 6
effect on wound healing and scar formation

| ID | Degree of healing |
|---|---|
| R14-R01 NON-TREATED | Moderate |
| R14-R05 NON-TREATED | Moderate* (*HF in scar tissue, cartilage |
| | reaction) |
| R14-L01 NON-TREATED | Moderate |
| Summary | The three scars are of similar nature. |
| R01-R02 Topiramate, aqueous | Technical problem with slide. Dermal |
| | defect not identified. No mound. |
| R01-R05 Topiramate, aqueous | Probable dermal defect (HF in center). |
| | No mound. |
| R01-L02 Placebo, aqueous | Dermal defect not identified. No mound. |
| Summary | Dermal defects are either not identified |
| | (R02 and L02) healing is Good as there is |
| | no mound and the fibrous tissue is |
| | relatively mature. |
| R03-R01 Topiramate, silicone | Good - no mound and relatively mature |
| | scar. |
| R03-R02 Topiramate, silicone | Technical problem with slide |
| R03-L02 Placebo, silicone | Good - no mound and relatively mature |
| | scar. |
| Summary | Placebo effect similar to treatment |
| R10-L01 Topiramate, nano- | Good - no mound and relatively mature |
| emulsion | scar. |
| R10-L05 Topiramate, nano- | Poor - mound is moderate but scar tissue |
| emulsion | less mature. |
| R10-R02 Placebo, nano- | Good - no mound and relatively mature |
| emulsion | scar |
| Summary | Placebo effect similar to treatment |

Scoring of healing:
If a slide is scored this implies that a dermal defect was identified.
Dermal defect - evidence that part of the skin has been excised. i.e a focus of continuous formation of fibrous tissue - scarring and absent HF.
Definitions:
(i) Poor
(ii) Moderate - a small mound composed of moderately mature scar tissue.
(iii) Good - no mound or sunk surface, mature scar tissue.

### Example 5

### Treatment of New Acne Scars

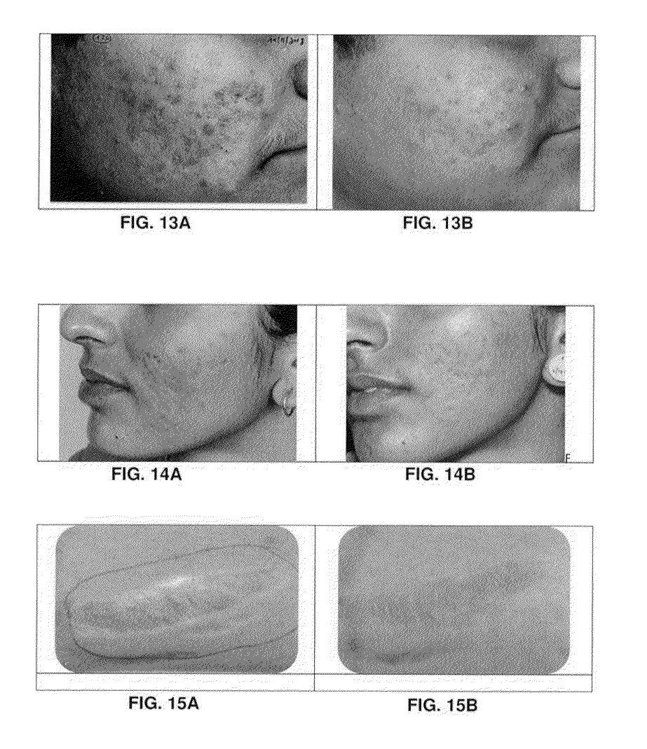

A 21 years old male patient, Indian, skin tone=Fitzpatrick 4, with inflammated post-Acne scars 3 months old, was treated once a day (evening time) for 3 weeks with a topical Topiramate 2.50% aqueous cream formulation. After 21 days of treatment, over 75% of improvement in Acne scar depth/severity and inflammation was observed (FIG. 13a, day 0—prior to treatment, FIG. 13b, 21 days post start of treatment).

A 24 years old female patient, Indian, skin tone=Fitzpatrick 3-4, with inflammated post-Acne scars 9 months old, was treated once a day (evening time) for 30 days with a topical Topiramate 2.50% aqueous cream formulation. After 30 days of treatment, over 50% of improvement in scar depth/severity and inflammation was observed (FIG. 14a, day 0—prior to treatment; FIG. 14b, 30 days post start of treatment). After several days of topical Topiramate application, the patient complained of occasional mild-to-moderate feeling of dryness in the facial skin following topical Topiramate application. The patient was instructed by the dermatologist to augment the topical Topiramate treatment with an over-the-counter moisturizer, and within 24 hours, the patient reported to the dermatologist that symptoms of dryness were resolved.

### Example 6

### Post-Steroids Skin Atrophy and Striae

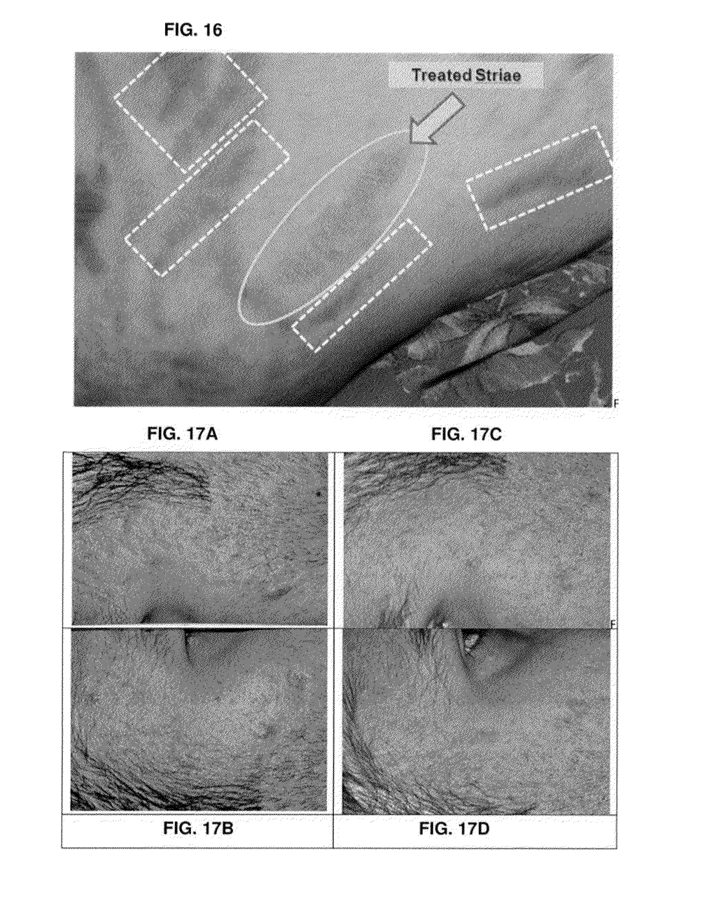

An 18 years old female patient, Indian, skin tone=Fitzpatrick 4-5, was continuously treated with a significant daily dosage of oral steroids for 12 months, causing whole body (75%+) skin atrophy and Striae down to the Fascia layer (FIG. 15a). Oral steroids were immediately discontinued, and out of hundreds of atrophy striae resulting from the steroidal treatment, a single, most deep and severe Striae atrophy was treated with topical Topiramate 2.50% once a day (evening) for a period of 8 weeks (FIG. 15b). The treated Striae atrophy was materially improved compared to other non-treated Striae atrophies (FIG. 16). The Dermis and Epidermis of the treated Atrophic Striae were fully regenerated, while the non-treated Atrophic Striae remained red, sunken and irregular.

After 60 days of treatment of the single most severe Striae atrophy (marked with an oval border, FIG. 16), both the Dermis and the Epidermis were recovered to their original level, and the Striae color tone became almost identical to the non-affected (healthy) skin color. In contrast, the non-treated Atrophic Striae (marked with a dotted rectangle border), remained sunken, irregular, with a color tone far more red and irregular comparing to the Atrophic Striae treated with topical Topiramate

### Example 7

### Mature Post-Acne Scars

A randomized, triple-blind study was conducted on 35 patients in two sites in India (Amritsar and Faridkot). Ethnic skin patients (Fitzpatrick 4-5) with mature atrophic post-Acne facial scarring (scar age >2 years) were randomly assigned to one of four study arms: topical Topiramate 5.0% (n=10), topical Topiramate 2.5% (n=9), oral Topiramate 25 mg (n=8) and topical placebo (n=7). Patients applied the topical formulation once a day on both cheeks for 3 months. Clinical assessment was performed at 6 weeks and 3 months using a visual analog scale (VAS) by the patients and 3 independent observers (2 plastic surgeons and 1 general surgeon) who scored side-by-side cheek photos (before and after treatment).

Thirty five patients completed the study, and a total of 57 cheeks (topical Topiramate 5%—n=18; topical Topiramate 2.5%—n=19; oral Topiramate—n=13; topical Placebo—n=7) were evaluated and scored.

Both topical Topiramate 2.50% and topical Topiramate 5.00% creams were well tolerated. At day 42, the improvement of the topical Topiramate 5.00% group was 30.26%, improvement in the topical Topiramate 2.50% group was 27.22%, improvement in the oral Topiramate group was 13.91%, and improvement in topical placebo group was 7.12% (representing advantage of the study group over the control of 325%, 282% and 95% respectively). At day 90, the improvement of the topical Topiramate 5.00% group was 45.93%, improvement in the topical Topiramate 2.50% group was 48.46%, improvement in the oral Topiramate group was 34.69%, and improvement in topical placebo group was 17.72% (representing an advantage of the study group over the control of 167%, 182% and 102% respectively). The results were statistically significant (p<0.05).

A female patient having a pre-study 20/20 vision which was enrolled in the oral Topiramate arm, suffered a transient reduction in vision to −5.00 within 2 days from starting on oral Topiramate (25 mg). Oral Topiramate treatment was immediately stopped by the Principal Investigator, and the patient's vision was fully resolved and returned to 20/20 within 7 days from stopping treatment. 35% of the patients treated with Topiramate cream (5.0% and 2.5%) complained about occasional mild-to-moderate feeling of facial skin dryness, which was resolved by using an over-the-counter commercial moisturizer. No other adverse events were reported in the topical drug or topical placebo groups.

### Example 8

### Treatment of Mature Atrophic Post-Acne Scarring

A 20 years old male patient, suffering from atrophic post-Acne scarring for 2 years, was treated with topical Topiramate 5.0% once a day (evening) for a period of 3 months (FIGS. 17a and 17b—prior to treatment; FIGS. 17c and 17d—90 days post start of treatment). 3 independent observers, which independently reviewed before and after images of each cheek treated without ever meeting or seeing the patient, provided an average score of 78% improvement of the patient's atrophic post-Acne scarring status.

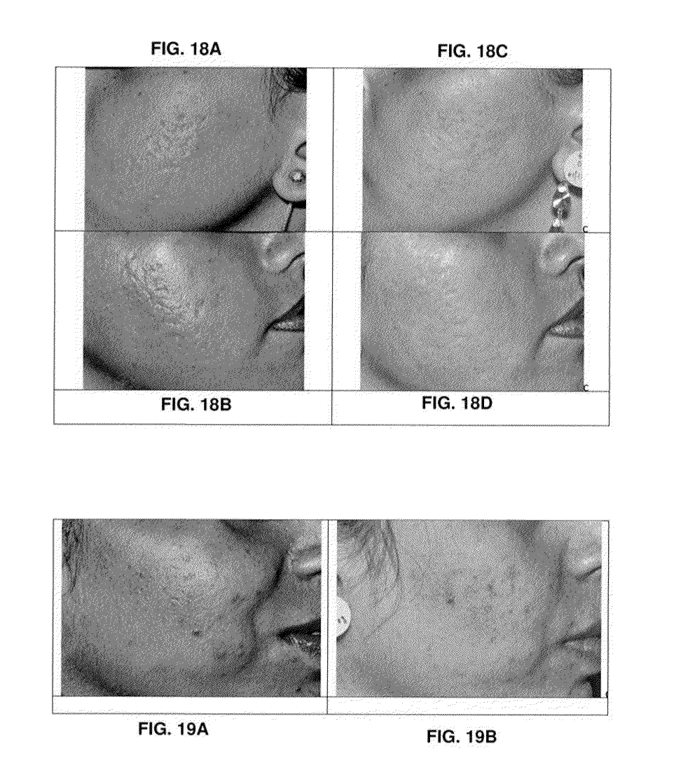

A 25 years old female patient, suffering from atrophic post-Acne scarring for 8 years, was treated with topical Topiramate 2.5% once a day (evening) for a period of 3 months (FIGS. 18a and 18b—prior to treatment; FIGS. 18c and 18d—90 days post start of treatment). 3 independent observers, which independently reviewed before and after images of each cheek treated without ever meeting or seeing the patient, provided an average score of 82% improvement in the patient's atrophic post-Acne scarring status.

### Example 9

### Prophylactic Treatment of Skin Aging

A 34 years old female patient, suffering from skin aging and atrophy symptoms for 5 years, was treated with topical Topiramate 5.0% once a day (evening) for a period 3 months (FIG. 19a—prior to treatment; FIG. 19b—90 days post start of treatment). An independent observation of skin health, vitality, thickness and roughness at the end of the 90 day treatment period revealed significant improvement of skin aging and atrophy symptoms. The skin looked significantly healthier, thicker, and was less rough to contact as compared to the pre-treatment baseline.

### Example 10

### Fresh, Post Cesarean Section Sutured Scar

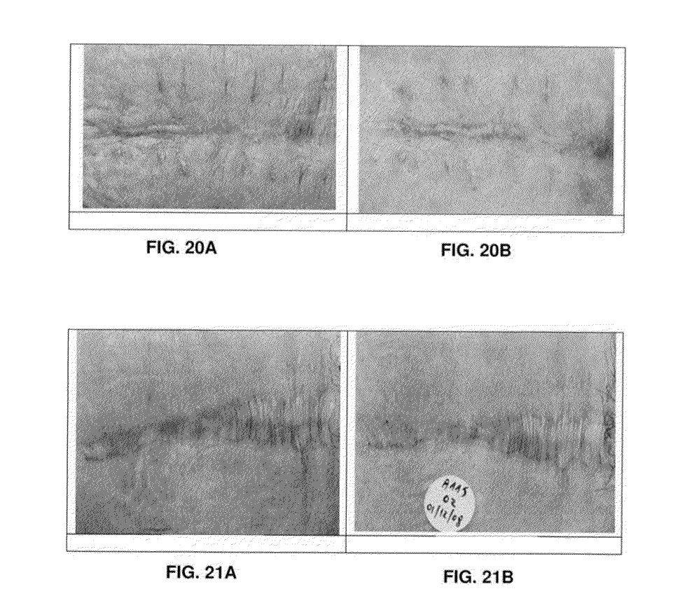

A female patient, Indian, 26 years old, with a 10 month-old semi-fresh post-Cesarean section scar, was treated once a day (evening) with topical Topiramate 5.00% for a period of 42 days (FIG. 20a—prior to treatment; FIG. 20b—42 days post start of treatment). The depth of atrophic scars resulting from side anchor suturing of the central Cesarean incision was significantly reduced and the irregular rough texture of skin around the atrophic scars was significantly improved over the 42 days of treatment.

### Example 11

### Mature, Post Cesarean Section Scar

A female patient, Indian, 24 years old, with a 24 month-old mature post-Cesarean section scar, was treated once a day (evening) with topical Topiramate 5.00% for a period of 42 days (FIG. 21a—prior to treatment; FIG. 21b—42 post start of treatment). The depth of the atrophic scar (left 50% of the suture incision scar) was significantly reduced and flattened over the 42 days of treatment.

### Example 12

### Treatment of Wrinkles and Stretch Marks

A study was conducted in order to evaluate the effect of once/twice daily topical topiramate treatment on skin wrinkles and stretch marks.

Facial wrinkles of 15 patients with an average age of 50 years were treated once/twice daily by topical application of a 1% or 1.5% Topiramate oil-in-water cream for 90 days. Stretch marks of 3 female patients with an average age of 35 years were also treated once/twice daily by topical application of a 1% or 1.5% Topiramate oil-in-water cream for 90 days.

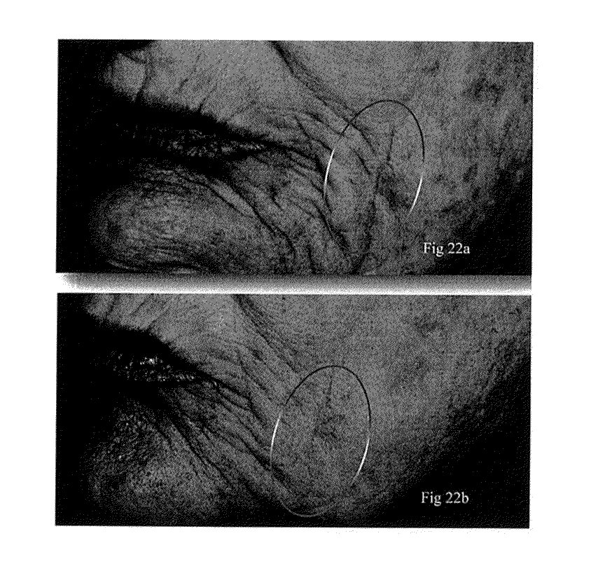

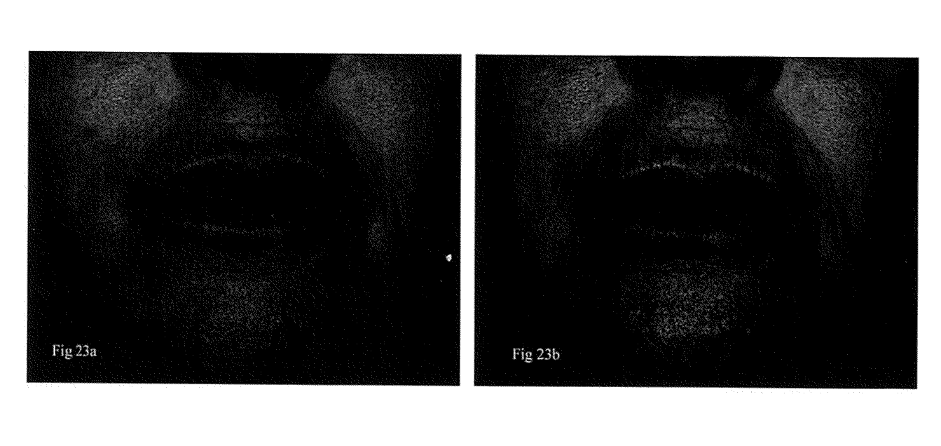

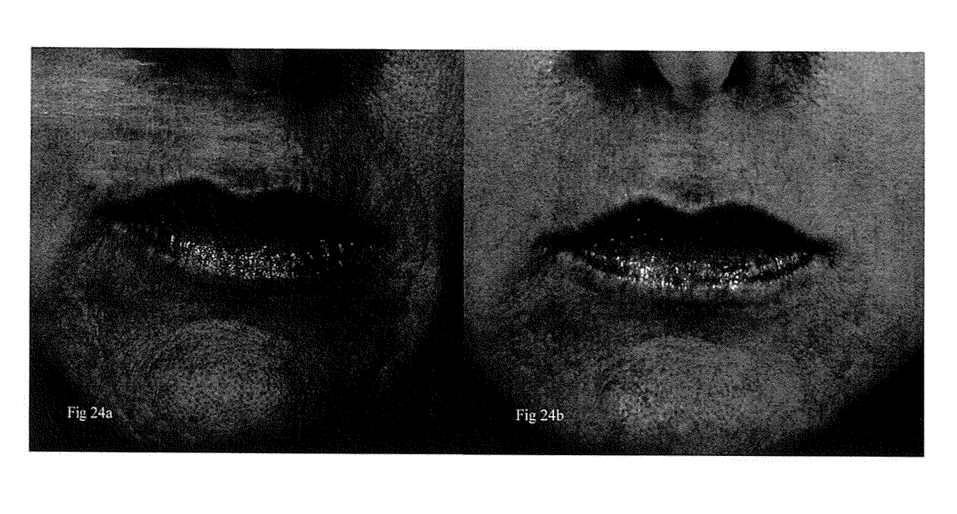

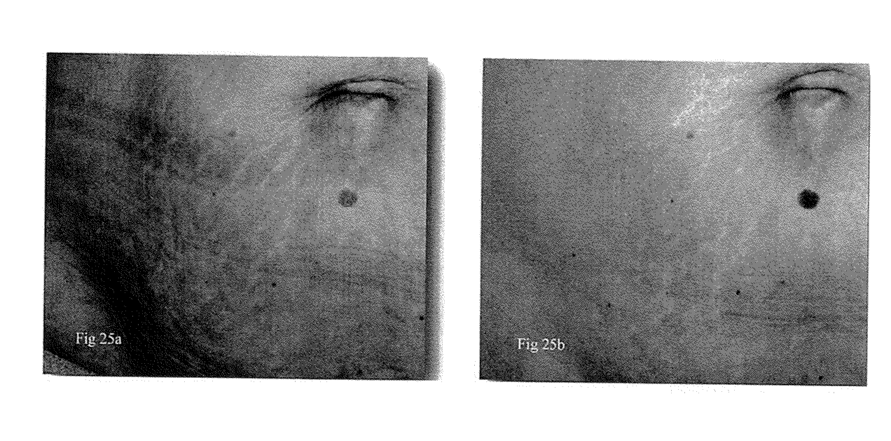

FIGS. 22-24 illustrate before and after images of individuals treated for facial wrinkles (a—before, b—after). As is shown by these Figures, once/twice daily application of topiramate cream substantially reduced the appearance of wrinkles over the 90 day treatment period (diminishment of wrinkles was observed as early as 30 days into treatment). FIG. 25 illustrate before and after images of an individual treated for stretch marks (a—before, b—after). As is shown by these Figures, once/twice daily application of topiramate cream substantially reduced the appearance of stretch marks over the 90 day treatment period (diminishment of stretch marks was observed as early as 45 days into treatment).

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A method of reducing the appearance of skin wrinkles or stretch marks comprising topically applying a pharmaceutical composition comprising topiramate and an oil-in-water carrier to said skin wrinkles or stretch marks thereby treating the disorder.

2. The method of claim 1, wherein said oil-in-water carrier is formulated as a cream, a gel cream an emulsion or a foam.

3. The method of claim 1, wherein said pharmaceutical composition includes 0.5-5.0% (w/w) of said topiramate.

4. The method of claim 1, wherein said pharmaceutical composition is applied once or twice daily for 30-90 days.
